(19) United States
(12) Patent Application Publication
(10) Pub. No.: US 2024/0270760 A1
(43) Pub. Date: Aug. 15, 2024

(54) GLYCERIC ACID, GLYCERATE, AND COMPOSITION THEREOF

(71) Applicants: INNER MONGOLIA KINGDOMWAY PHARMACEUTICAL CO., LTD., Huhhot (CN); XIAMEN KINGDOMWAY GROUP COMPANY, Xiamen (CN); KINGDOMWAY BIOTECHNOLOGY (JIANGSU) CO., LTD., Nantong (CN)

(72) Inventors: Ming ZHEN, Huhhot (CN); Mengjing LIU, Huhhot (CN); Li WANG, Huhhot (CN); Qiyue LIU, Nantong (CN); Biqin CHEN, Xiamen (CN); Bingrong WANG, Xiamen (CN); Dan LI, Xiamen (CN); Yi WU, Xiamen (CN); Guanghuang ZHAN, Xiamen (CN)

(73) Assignees: INNER MONGOLIA KINGDOMWAY PHARMACEUTICAL CO., LTD., Huhhot (CN); XIAMEN KINGDOMWAY GROUP COMPANY, Xiamen (CN); KINGDOMWAY BIOTECHNOLOGY (JIANGSU) CO., LTD., Nantong (CN)

(21) Appl. No.: 18/594,000

(22) Filed: Mar. 4, 2024

Related U.S. Application Data
(63) Continuation of application No. PCT/CN2023/106405, filed on Jul. 7, 2023.

(30) Foreign Application Priority Data
Feb. 8, 2023 (CN) 202310080043.4

(51) Int. Cl. C07F 3/04

(52) U.S. Cl. CPC C07F 3/04; C07B 2200/13

(57) ABSTRACT

The present disclosure provides a crystalline form of calcium D-glycerate of formula

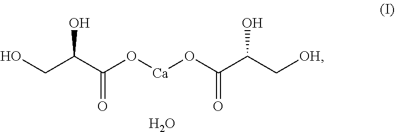

and a method for preparing the crystalline form of calcium D-glycerate of formula (I). The crystalline form may be characterized by having X-ray powder diffraction pattern (XRPD) diffraction peaks (2θ degrees) at 12.9°±0.2°, 20.9°±0.2°, and 31.7°±0.2°.

### CROSS-REFERENCE TO RELATED APPLICATIONS

This application is a continuation of International Application No. PCT/CN2023/106405, filed on Jul. 7, 2023, which claims the priory of Chinese Patent Application No. 202310080043.4 filed on Feb. 8, 2023, the contents of which are hereby incorporated by reference.

### TECHNICAL FIELD

The present disclosure relates to the field of pharmaceutical chemistry, and in particular to glyceric acid, glycerate, and composition thereof.

### BACKGROUND

Glyceric acid and/or glycerate play an active role in human metabolism. For example, glyceric acid and/or glycerate in the body can accelerate the oxidation of acetaldehyde, thereby accelerating the metabolism of ethanol in the body. These compounds have various applications that can benefit human health. Therefore, it is desirable to provide effective and efficient approaches to produce glyceric acid and/or glycerate, in different forms and compositions thereof.

### SUMMARY

One aspect of the present disclosure provides a crystalline form of calcium D-glycerate of formula (I)

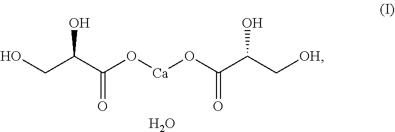

wherein the crystalline form is characterized by having X-ray powder diffraction pattern (XRPD) diffraction peaks (2θ degrees) at 12.9°±0.2°, 20.9°±0.2°, and 31.7°±0.2°.

Another aspect of the present disclosure provides a method for preparing crystalline form of calcium D-glycerate of the formula (I). The method may include mixing D-glyceric acid with a calcium salt to form a first solution; stirring the first solution at a stirring rate and cooling the first solution to a temperature at a cooling rate; aging the first solution for a time period at the temperature; filtering the first solution to obtain a first calcium glycerate sample and drying the first calcium glycerate sample; redissolving the dried first calcium glycerate sample to form a second solution at a second temperature for a second time period; and filtering the second solution to obtain a second calcium glycerate sample and drying the second calcium glycerate sample to obtain the crystalline form of calcium glycerate.

Another aspect of the present disclosure provides method for preparing crystalline form of calcium D-glycerate of the formula (I). The method may include mixing D-glyceric acid with a calcium salt to form a first solution; adding a solvent into the first solution and stirring at a stirring rate to form a second solution; aging the second solution for a time period; filtering the second solution to obtain a first calcium glycerate sample and drying the first calcium glycerate sample; redissolving the dried first calcium glycerate sample to form a third solution; and filtering the third solution to obtain a second calcium glycerate sample and drying the second calcium glycerate sample to obtain the crystalline form of calcium glycerate.

Another aspect of the present disclosure provides a crystalline form of calcium D-glycerate of formula (II)

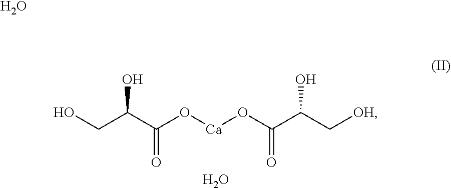

wherein the crystalline form is characterized by having X-ray powder diffraction pattern (XRPD) diffraction peaks (2θ degrees) at 15.4°±0.2°, 17.0°±0.2°, and 27.1°±0.2°.

Another aspect of the present disclosure provides a method for preparing crystalline form of calcium D-glycerate of the formula (II). The method may include mixing D-glyceric acid with a calcium salt to form a first solution; adjusting pH of the first solution to a pH range; adding methyl alcohol into the first solution to form a second solution; refluxing the second solution at a first temperature for a first time period; stopping refluxing, and stirring and cooling the second solution naturally; after cooling the second solution to a second temperature, adding a solvent into the second solution and stirring to form a third solution; aging the third solution for a second time period; and filtering the third solution to obtain calcium glycerate sample and drying the calcium glycerate sample to obtain the crystalline form of calcium glycerate.

Another aspect of the present disclosure provides a method for preparing an amorphous form of calcium glycerate of formula (III)

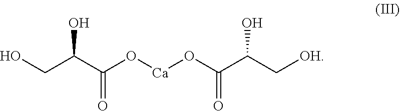

The method may include stirring a crystalline form of calcium glycerate with a solvent at a first temperature for a first time period to form a suspension solution; maintaining the first temperature and filtering the suspension solution to form a supersaturated solution; stirring the supersaturated solution at a stirring rate, and cooling the supersaturated solution at a cooling rate; after cooling the supersaturated solution to a second temperature, maintaining the second temperature, aging the supersaturated solution for a second time period, and filtering the supersaturated solution; and drying the filtered product for a third time period at a pressure and a third temperature to obtain the amorphous form of calcium glycerate.

Another aspect of the present disclosure provides a method for preparing an amorphous form of calcium glycerate of the formula (III). The method may include stirring a crystalline form of calcium glycerate with a solvent at a first temperature for a first time period to form a suspension solution; maintaining the first temperature and filtering the suspension solution to form a supersaturated solution; stirring the supersaturated solution at a stirring rate, and cooling the supersaturated solution at a cooling rate; after cooling the supersaturated solution to a second temperature, maintaining the second temperature, aging the supersaturated solution for a second time period, and filtering the supersaturated solution; and drying the filtered product for a third time period at a pressure and a third temperature to obtain the amorphous form of calcium glycerate.

Another aspect of the present disclosure provides a method for preparing a purified glyceric acid sample. The method may include obtaining a fermentation broth of glyceric acid based on a microbial fermentation technique; performing a solid-liquid separation operation to the fermentation broth to filter out a first impurity from the fermentation broth, the first impurity including bacteria residues, cell residues, and a macromolecular substance; filtering out a second impurity from the fermentation broth using a magnetic nano adsorbent and an aluminum/ferrum coagulating agent, the second impurity including protein, tannin, sticky sugar, and pigment; and performing an extraction operation to filter out a third impurity from the fermentation broth to obtain the purified glyceric acid sample, the third impurity including glycerin and dihydroxyacetone.

Another aspect of the present disclosure provides a composition. The composition may include a glycerate substance, including D-glyceric acid or a salt of D-glyceric acid; and an excipient including at least one of an NAD+ precursor, a mineral, or a vitamin.

### BRIEF DESCRIPTION OF THE DRAWINGS

FIG. 1 shows an X-ray powder diffraction pattern (XRPD) pattern of crystalline Form I according to some embodiments of the present disclosure;

FIG. 2 shows a differential scanning calorimetry (DSC) spectrum of crystalline Form I according to some embodiments of the present disclosure;

FIG. 3 shows a differential thermal analysis (DTA) spectrum of crystalline Form I according to some embodiments of the present disclosure;

FIG. 4 shows an XRPD pattern of crystalline Form II according to some embodiments of the present disclosure;

FIG. 5 shows a DSC spectrum of crystalline Form II according to some embodiments of the present disclosure;

FIG. 6 shows a DTA spectrum of crystalline Form II according to some embodiments of the present disclosure;

FIG. 7 shows an XRPD pattern of crystalline Form III according to some embodiments of the present disclosure;

FIG. 8 shows a change of crystalline Form I and crystalline Form II in terms of percentage of total compound over 6 months at a temperature of 40±2° C. and a humidity of 75%, providing estimates of the stability of these forms, according to some embodiments of the present disclosure;

FIG. 9 shows the dissolution of calcium glycerate of different forms in water at 37±0.2° C. according to some embodiments of the present disclosure;

FIG. 10 illustrates a method for preparing a purified glyceric acid sample according to some embodiments of the present disclosure;

FIG. 11 shows an adsorption result of protein with respect to the pH of the protein solution according to some embodiments of the present disclosure;

FIG. 12 shows a Fourier transform infrared spectrum characterization of a coupling reaction according to some embodiments of the present disclosure;

FIG. 13 shows transmission scanning electron microscope characterization of nanoparticle sizes of pure Fe₃O₄ and a magnetic nano adsorbent according to some embodiments of the present disclosure;

FIG. 14 shows transmission scanning electron microscope characterization of flocs according to some embodiments of the present disclosure;

FIG. 15 shows a sample of calcium glycerate according to some embodiments of the present disclosure;

FIG. 16 shows a high-performance liquid chromatography (HPLC) spectrum of glyceric acid according to some embodiments of the present disclosure;

FIG. 17 shows an HPLC spectrum of calcium glycerate according to some embodiments of the present disclosure; and

FIG. 18 shows a Fourier transform infrared spectrum characterization of calcium glycerate according to some embodiments of the present disclosure.

### DETAILED DESCRIPTION

In the following detailed description, numerous specific details with reference to the accompanying drawings are set forth by way of examples in order to provide a thorough understanding of the relevant disclosure. Various modifications to the disclosed embodiments will be readily apparent to those skilled in the art, and the general principles defined herein may be applied to other embodiments and applications without departing from the spirit and scope of the present disclosure. The identical numerals in the drawings represent the same or similar structures or operations, unless the context clearly indicates otherwise.

It will be understood that the term “system,” “device,” “unit,” and/or “module,” used herein are one method to distinguish different components, elements, parts, sections or assemblies of different levels in ascending order. However, the terms may be displaced by another expression if they achieve the same purpose.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

The range of values used herein in the present disclosure briefly illustrates each value in the range of values.

The flowcharts used in the present disclosure illustrate operations that systems implement according to some embodiments of the present disclosure. It should be noted that the foregoing or the following operations may not be performed in the order accurately. Instead, the steps may be processed in reverse order or simultaneously. Besides, one or more other operations may be added to the flow charts, or one or more operations may be omitted from the flow chart.

Some embodiments of the present disclosure may provide a crystalline form of calcium D-glycerate (also referred to as crystalline Form (I) of formula

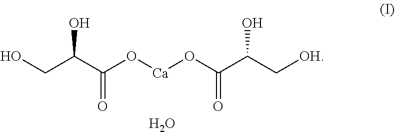

In some embodiments, by using Cu-Kα radiation, crystalline Form I may be characterized by having X-ray powder diffraction pattern (XRPD) diffraction peaks (20 degrees) at 12.9°±0.2°, 20.9°±0.2°, and 31.7°±0.2°.

In some embodiments, crystalline Form I may be further characterized by having XRPD diffraction peaks (2θ degrees) at 25.4°±0.2° and 26.1°±0.2°.

In some embodiments, crystalline Form I may be further characterized by having XRPD diffraction peaks (2θ degrees) at 15.6°±0.2°, 16.6°±0.2°, 18.8°±0.2°, 22.3°±0.2°, 23.1°±0.2°, 27.4°±0.2°, 27.8°±0.2°, 33.3°±0.2°, 35.2±0.2°, and 38.2°±0.2°.

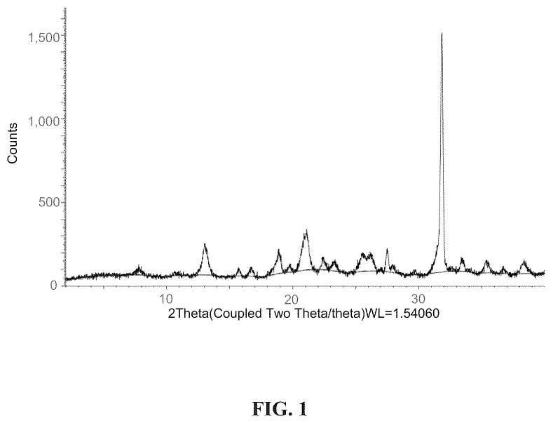

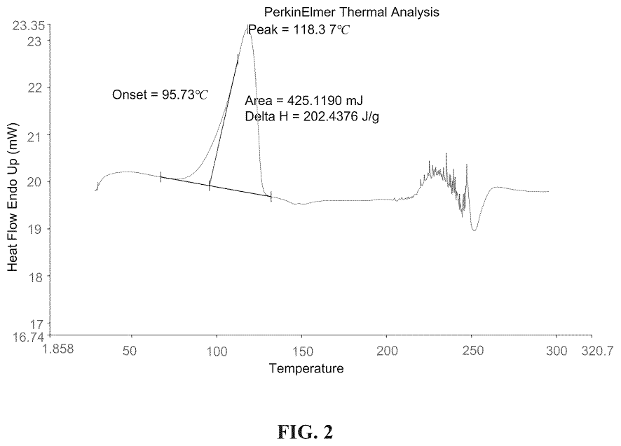

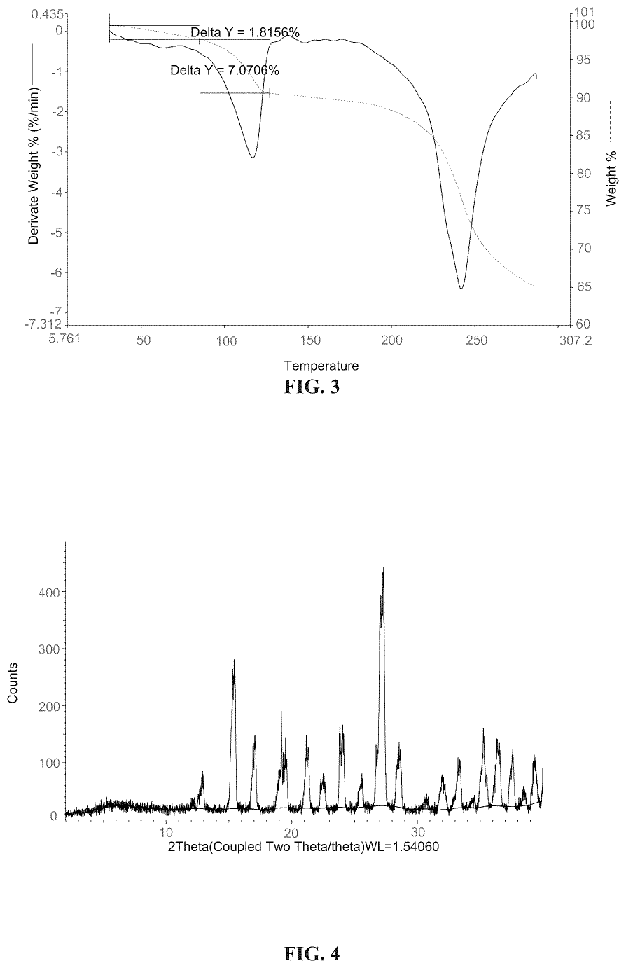

In some embodiments, crystalline Form I may have XRPD as shown in FIG. 1. Crystalline Form I may have a DSC spectrum as shown in FIG. 2. Crystalline Form I may have a DTA spectrum as shown in FIG. 3.

According to some embodiments of the present disclosure, a method for preparing Form I may be provided. The method may include the following steps.

Step (1): mixing D-glyceric acid with a calcium salt to form a first solution;

In some embodiments, a D-glyceric acid extraction solution may be concentrated, and the calcium salt may be added to the concentrated solution at a temperature and completely dissolved by stirring to form the first solution. In some embodiments, the temperature may range from 20° C. to 60° C., 30° C. to 50° C., etc. In some embodiments, the purity of the D-glyceric acid extraction solution may range from 85% to 100%, from 95% to 100%, etc. In some embodiments, the D-glyceric acid extraction solution may be concentrated at a temperature ranging from 20° C. to 65° C., from 40° C. to 50° C., etc. In some embodiments, a molar ratio of the calcium salt to D-glyceric acid may range from 0.5 to 0.53. In some embodiments, the content of the concentrated solution may range from 100 g/L to 300 g/L, from 150 g/L to 250 g/L, etc. In some embodiments, the calcium salt may include calcium chloride, for example, anhydrous calcium chloride, dihydrate calcium chloride, tetrahydrate calcium chloride, hexahydrate calcium chloride, or the like, or a mixture thereof.

Step (2): stirring the first solution at a stirring rate and cooling the first solution to a temperature at a cooling rate; aging the first solution for a time period at the temperature; and filtering the first solution to obtain a first calcium glycerate sample and drying the first calcium glycerate sample;

In some embodiments, the stirring rate may range from 10 r/min to 40 r/min or from 20 r/min to 25 r/min. In some embodiments, the cooling rate may range from 3° C./h to 8° C./h, from 4° C./h to 6° C./h, etc. In some embodiments, the temperature may range from 3° C. to 20° C., from 5° C. to 15° C., etc. In some embodiments, the time period may range from 0.25 h to 8 h, from 2 h to 4 h, etc. In some embodiments, step (2) may be performed one or more times. For example, step (2) may be performed once.

Step (3): redissolving the dried first calcium D-glycerate sample to form a second solution; filtering the second solution to obtain a second calcium D-glycerate sample and drying the second calcium glycerate sample to obtain the crystalline form of calcium glycerate (i.e., crystalline Form (I)).

In some embodiments, the dried first calcium D-glycerate sample may be redissolved in water to form the second solution. A weight ratio of water to the dried first calcium D-glycerate sample may range from 3 to 10, 5 to 8, etc. In some embodiments, the dried first calcium D-glycerate sample may be redissolved at a second temperature for a second time period. The second temperature may range from 20° C. to 60° C., from 35° C. to 50° C., etc. The second time period may range from 0.2 h to 2 h, from 0.5 h to 1 h, etc.

It should be noted that the above descriptions are for illustration purposes and non-limiting. In some alternative embodiments, step (2) may be performed by other manners. For example, step (2) may include: adding a solvent into the first solution and stirring at a stirring rate to form a second solution; aging the second solution for a time period; filtering the second solution to obtain a first calcium glycerate sample and drying the first calcium glycerate sample. In some embodiments, the solvent may include methanol, ethanol, isopropanol, glycerol, 1,2-propanediol, 1,3-propanediol, ethylene glycol, or the like, or a mixture thereof. In some embodiments, a volume ratio of the solvent to the first solution may range from 1/6 to 3, from 1/3 to 1, etc. In some embodiments, the stirring rate may range 10 r/min to 40 r/min from 25 r/min to 35 r/min, etc. In some embodiments, the time period may range from 0.25 h to 4 h, from 1 h to 2 h, etc.

Some embodiments of the present disclosure may provide a crystalline form of calcium D-glycerate (also referred to as crystalline Form II) of formula

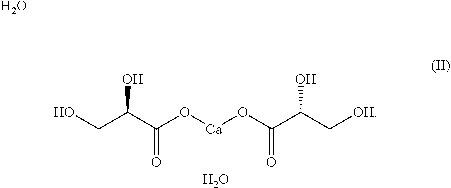

In some embodiments, by using Cu-Kα radiation, crystalline Form II may be characterized by having XRPD diffraction peaks (2θ degrees) at 15.4°±0.2°, 17.0°±0.2°, and 27.1°±0.2°.

In some embodiments, crystalline Form II may be further characterized by having XRPD diffraction peaks (2θ degrees) at 23.8°±0.2°, 28.6°±0.2°, and 32.0°±0.2°.

In some embodiments, crystalline Form II may be further characterized by having XRPD diffraction peaks (2θ degrees) at 12.9°±0.2°, 19.1°±0.2°, 19.5°±0.2°, 21.2°±0.2°, 22.3°±0.2°, 36.4°±0.2°, 37.6°±0.2°, and 39.5°±0.2°.

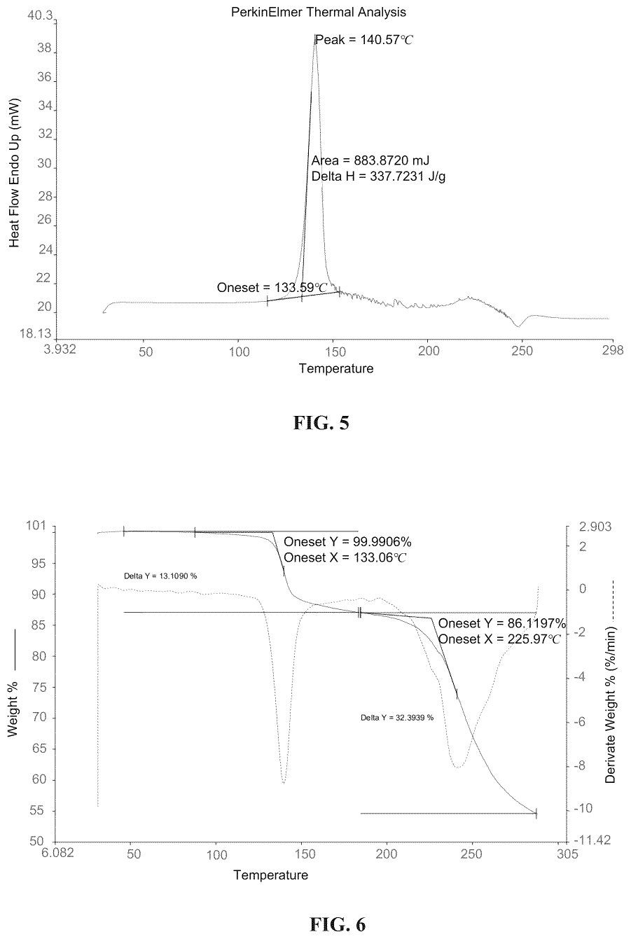

In some embodiments, crystalline Form II may have an XRPD pattern as shown in FIG. 4. Crystalline Form II may have a DSC spectrum as shown in FIG. 5. Crystalline Form II may a DTA spectrum as shown in FIG. 6. Peak areas in FIG. 5 and FIG. 6 indicate that crystalline Form II loses water of crystallization in a temperature range from 125 to 150° C., and the weight of water loss corresponds to two molecules of water of crystallization.

According to some embodiments of the present disclosure, a method for preparing crystalline Form II may be provided. The method may include the following steps.

Step (1): D-glyceric acid with a calcium salt may be mixed to form a first solution.

In some embodiments, a D-glyceric acid extraction solution may be concentrated, and the calcium salt may be added to the concentrated solution and completely dissolved by stirring to form the first solution. In some embodiments, D-glyceric acid and the calcium salt may be mixed at a temperature ranging from 20° C. to 60° C. or from 30° C. to 50° C., etc. In some embodiments, the purity of the D-glyceric acid extraction solution may range from 85% to 100%, from 95% to 100%, etc. In some embodiments, the D-glyceric acid extraction solution may be concentrated at a temperature ranging from 20° C. to 65° C., from 40° C. to 50° C., etc. In some embodiments, a molar ratio of the calcium salt to D-glyceric acid may range from 0.5 to 0.53. In some embodiments, the content of the concentrated solution may range from 1009 to 3001, from 1509 to 2509, etc. In some L L‘ L L’ embodiments, the calcium salt may include calcium chloride, for example, anhydrous calcium chloride, dihydrate calcium chloride, tetrahydrate calcium chloride, hexahydrate calcium chloride, or the like, or a mixture thereof.

Step (2): the pH of the first solution may be adjusted to a pH range; methyl alcohol may be added to the first solution to form a second solution; and the second solution may be refluxed at a first temperature for a first time period.

In some embodiments, the pH range may include a range from 2.5 to 5.8 from 3.0 to 4.0, etc. In some embodiments, a volume ratio of methyl alcohol to the first solution may range from 0.3 to 1, from 0.5 to 0.8, etc. In some embodiments, the first temperature may range from 60° C. to 80° C., from 65° C. to 78° C., etc. In some embodiments, the first time period may range from 20 min to 120 min, from 40 min to 80 min, etc.

Step(3): the refluxing may be stopped, and the second solution may be stirred and cooled naturally; after the second solution is cooled to a second temperature, a solvent may be added to the second solution and stirred to form a third solution; the third solution may be aged for a second time period; and the third solution may be filtered to obtain calcium glycerate sample and the calcium glycerate sample may be dried to obtain the crystalline form of calcium glycerate (i.e., crystalline Form (II)).

In some embodiments, the second temperature may range from 10° C. to 40° C., from 15° C. to 25° C., etc. In some embodiments, a volume ratio of the solvent to the first solution may range from 0.3 to 2, from 0.8 to 1, etc. In some embodiments, the solvent may include methanol, ethanol, or the like, or any mixture thereof. In some embodiments, the adding the solvent into the second solution may maintain for a time period ranging from 0.5 h to 3 h, from 1 h to 2 h, etc. In some embodiments, the second time period may range from 0.5 h to 4 h, from 1 h to 2 h, etc.

Some embodiments of the present disclosure may provide a method for preparing an amorphous form of calcium glycerate (also referred to as crystalline Form III)

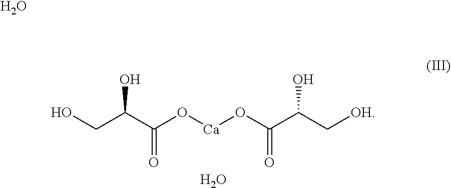

Step (1): a crystalline form of calcium glycerate (e.g., crystalline Form I, crystalline Form II) with a solvent at a first temperature for a first time period may be stirred to form a suspension solution, the first temperature may be maintained and the suspension solution may be filtered to form a supersaturated solution.

In some embodiments, the first temperature may range from 20° C. to 60° C., from 40° C. to 50° C., etc. In some embodiments, the solvent may include methanol, ethanol, acetonitrile, acetone, dimethyl sulfoxide, or N—N dimethylformamide, or the like, or a mixture thereof. In some embodiments, the first time period may range from 20 min to 120 min, from 60 min to 90 min, etc.

Step (2): the supersaturated solution may be stirred at a stirring rate, and the supersaturated solution may be cooled at a cooling rate; after the supersaturated solution is cooled to a second temperature, the second temperature may be mained, the supersaturated solution may be aged for a second time period, and the supersaturated solution may be filtered; and the filtered product may be dried for a third time period at a pressure and a third temperature to obtain the amorphous form of calcium glycerate.

In some embodiments, the stirring rate may range from 40 r/min to 120 r/min, from 60 r/min to 100 r/min, etc. In some embodiments, the cooling rate may range from 40° C./h to 90° C./h, from 60° C./h to 80° C./h, etc. In some embodiments, the second temperature may range from −10° C. to 10° C. or from 0° C. to 8° C., etc. In some embodiments, the second time period may range from 1 h to 10 h, from 3 h to 5 h, etc. In some embodiments, the pressure may range from −0.07 MPa to 0.10 MPa, from −0.08 MPa to 0.09 MPa, etc. In some embodiments, the third temperature may range from 20° C. to 50° C. or from 30° C. to 40° C., etc. In some embodiments, the third time period may range from 1 h to 8 h or from 3 h to 5 h, etc.

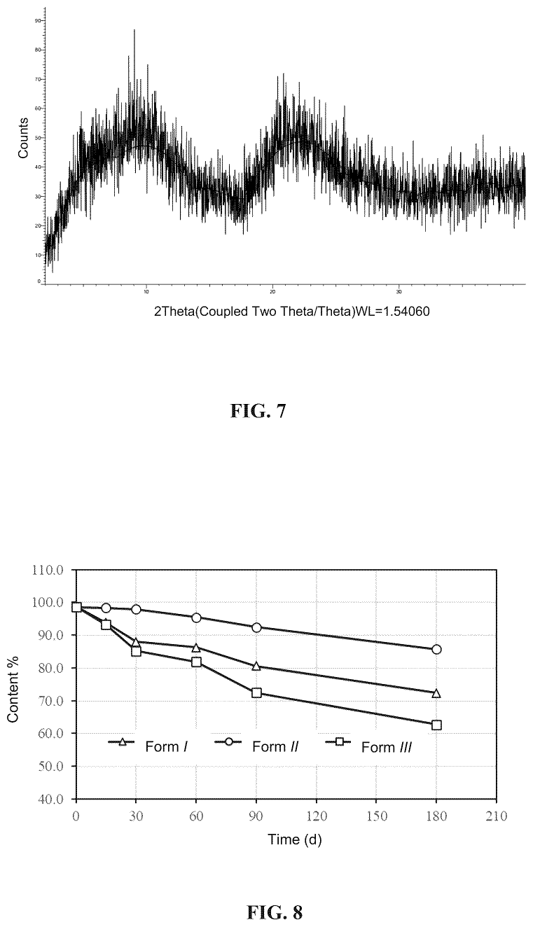

In some embodiments, by using Cu-Kα radiation, crystalline Form III has an XRPD pattern as shown in FIG. 7, which indicates that Form III is amorphous.

In some embodiments, FIG. 8 shows a change of crystalline Form I and crystalline Form II in terms of percentage of total compound over 6 months at a temperature of 40±2° C. and a humidity of 75%, providing emstimates of the stability of these forms. As indicated by FIG. 8, the thermal stability of crystalline Form II is better than the thermal stability of crystalline Form I and crystalline Form III. The thermal stability of crystalline Form I is better than the thermal stability of crystalline Form III.

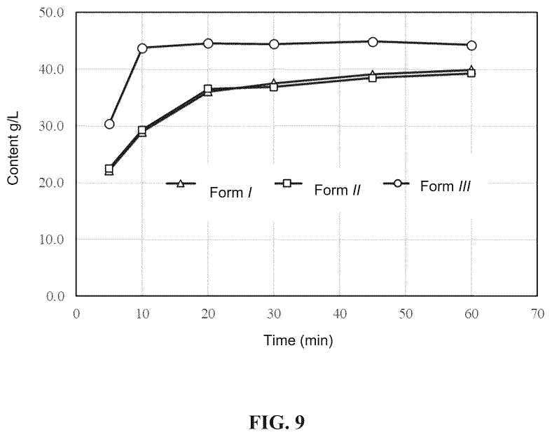

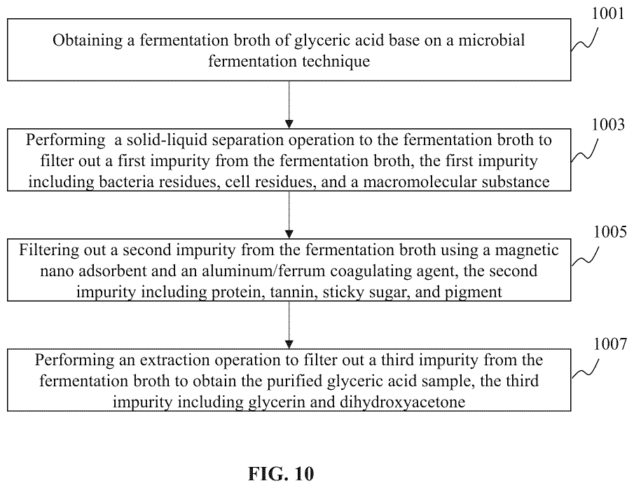

In some embodiments, FIG. 9 shows the dissolution of calcium glycerate of different forms in water at 37±0.2° C. As indicated by FIG. 9, the dissolution rate of crystalline Form III in water at 37±0.2° C. is faster than that of crystalline Form I. The dissolution rate of crystalline Form II in water at 37±0.2° C. is faster than that of crystalline Form I.

### Example 1: Preparation Method of Crystalline Form I

500 ml of a D-glyceric acid extraction solution with a purity of 96.8% was concentrated to obtain a concentrated solution of 200 g/L under a reduced pressure (e.g., from −0.06 MPa to 0.09 MPa) at 45° C. 5.23 g of anhydrous calcium chloride was added to the concentrated solution and stirred at about 40° C. until completely dissolved to obtain a sample. The stirring speed was maintained at 35 r/min. The cooling rate of the sample was controlled at 5° C./h and the temperature of the sample is decreased to 8° C. to obtain a cooled sample. The cooled sample was aged for 3 h to obtain an aged sample. The aged sample was filtered and dried to obtain a dried sample. The dried sample (i.e., the dried D-calcium glycerate) was dissolved in water with 6 times the volume at 45° C. for 1 hour to obtain a dissolved sample. The stirring speed of the dissolved sample was adjusted to 25 r/min, the cooling rate of the dissolved sample was adjusted to 6° C./h, and the temperature of the dissolved sample was decreased to 5° C. to obtain a cooled dissolved sample. The cooled dissolved sample was aged for 4 hours to obtain an aged dissolved sample. The aged dissolved sample was filtered and dried to obtain a sample of crystalline Form I.

### Example 2: Preparation Method of Crystalline Form I

4 L of a D-glyceric acid extraction solution with a purity of 97.2% was concentrated to obtain a concentrated solution of 230 g/L at 50° C. under a reduced pressure (e.g., from −0.06 MPa to 0.09 MPa). 55.47 g of calcium chloride dihydrate was added to the concentrated solution and stirred at 45° C. until completely dissolved to obtain a mixture. 1 volume times of ethanol was added to the mixture and stirred at 35 r/min to obtain a sample. The sample was aged for 1 h to obtain an aged sample. The aged sample may be filtered and dried to obtain a tried sample (i.e., dried D-glyceride calcium). 4 times of water was added to the dried sample to dissolve the dried D-glyceride calcium at 40° C. for 1 hour to obtain a dissolved sample. The stirring speed of the dissolved sample was adjusted to 30 r/min. Ethanol with once (i.e., one time) the volume was slowly added to the dissolved sample, and stirred at 25 r/min to obtain a second dissolved sample. The second dissolved sample was aged for 1.5 hours to obtain a second dried sample. The second dried sample was filtered and dried to obtain a sample of crystalline Form I.

### Example 3: Preparation Method of Crystalline Form II

2 L of a D-glyceric acid extraction solution with a purity of 98.0% was concentrated to 180 g/L at 50° C. under a reduced pressure (e.g., from −0.06 MPa to 0.09 MPa) to obtain a concentrated solution. 20.34 g of anhydrous calcium chloride was added to the concentrated solution, and stirred at 50° C. until completely dissolved to obtain a sample. A small amount of hydrochloric acid was added to the sample to adjust the pH of the sample to 3.6. 150 ml of methanol was added to the PH-adjusted sample to obtain a dissolved sample. The methanol sample was heated up to 65° C. and refluxed for 60 min. The refluxing was stopped to obtain a refluxed sample. The refluxed sample was stirred to cool down naturally to obtain a cooled sample which is cooled to 15° C. 300 ml of methanol was added to the cooled sample within 1 hour to obtain a second dissolved sample. The stirring speed was controlled to 30 r/min. The second dissolved sample was aged for 2 hours to obtain an aged sample. The aged sample was filtered and dried to obtain a sample of crystalline Form II.

### Example 4: Preparation Method of Crystalline Form III

20 g of calcium D-glycerate of crystalline Forms I or II was added to 100 ml of anhydrous methanol and stirred at 45° C. for 60 minutes to form a suspension solution. The suspension solution was filtered while the temperature was maintained to obtain a filtered solution. The filtered solution was maintained and stirred at 90 r/min, rapidly cooled down at a speed of 70° C./h, and the temperature was maintained when the temperature dropped to 5±0.2° C. to obtain a cooled sample. The cooled sample was aged for 5 h and filtered while the temperature was maintained to obtain a filtered sample. The filtered sample was dried at 35° C. and −0.085 MPa for 4 hours to obtain a sample of crystalline Form III.

According to some embodiments of the present disclosure, a method for preparing a purified glyceric acid sample may be provided.

In 1001, a fermentation broth of glyceric acid may be obtained based on a microbial fermentation technique.

In addition to glyceric acid (i.e., the target product), the fermentation broth may also include impurities, e.g., soluble substances such as dihydroxyacetone, glycerol, insoluble substances such as bacteria, cell residues, macromolecular substances, etc.

In 1003, a solid-liquid separation operation (e.g., centrifugation, filtration) may be performed on the fermentation broth to filter out a first impurity from the fermentation broth. The first impurity may include bacteria residues, cell residues, macromolecular substances, etc. The bacteria and/or cell residues may continue to ferment and even grow other microorganisms, resulting in the deterioration of the fermentation broth. Therefore, the bacteria and/or cell residues should be removed within a relatively short time period after the obtainment of the fermentation broth, so that the fermentation broth may be not easily contaminated by the bacteria and/or deteriorate.

Due to the high viscosity of the fermentation broth, the bacteria and/or cell residues may be filtered out via the solid-liquid separation operation. In addition, the macromolecular substances may also be filtered out via the solid-liquid separation operation. In some embodiments, the solid-liquid separation operation may be achieved by a ceramic membrane equipment. A pore range of the ceramic membrane equipment may range from 50 nm to 300 nm, 200 nm, etc. A pressure range of the filtration may be controlled at a range from 0.1 MPa to 1.0 MPa, 0.3 MPa, etc.

After the solid-liquid separation operation, a filtration solution may be obtained. The solids/moisture content of the filtration solution may be lower than 2%.

In 1005, a second impurity may be filtered out from the fermentation broth (e.g., the filtration solution described in 1003) using a magnetic nano adsorbent and an aluminum/ferrum coagulating agent. The second impurity may include natural organic substances such as protein, tannin, sticky sugar, pigment, etc.

In some embodiments, step 1005 may include two stages: adding the magnetic nano adsorbent into the filtration solution to filter out the second impurity (also referred to as stage 1), and adding the aluminum/ferrum coagulating agent into the filtration solution to conjugate with the magnetic nano adsorbent to further filter out the second impurity (also referred to as stage 2). The conjugated substance may also be referred to as a magnetic nano-aluminum/ferrum adsorbent.

In some embodiments, the amount of the magnetic nano adsorbent may be 0.1%-10% (e.g., 2%) of the volume of the filtration solution. In some embodiments, the stirring rate of stage 1 may be controlled at 30 r/h-200 r/h (e.g., 80 r/h). In some embodiments, the temperature of stage 1 may be controlled at 10° C. to 50° C. (e.g., 25° C.). In some embodiments, stage 1 may maintain for a time period, for example, 0.5 hours-5 hours (e.g., 2 hours).

In some embodiments, the magnetic nano adsorbent used in stage 1 may be prepared by: using Fe₃O₄ as the magnetic core, using the surfactant polyethylene glycol 6000 as the iron salt dispersant to improve the surface performance of the magnetic nano adsorbent and reduce the agglomeration of the magnetic nano-particles, and further using attapulgite as the surface shell material. A chemical co-precipitation technique may be used to prepare the magnetic nano adsorbent with a small particle size, uniform size, and good dispersion.

In some embodiments, the magnetic nano adsorbent used in stage 1 may be prepared by following operations.

(1) A Ferrum Salt May be Mixed with a Solvent to Prepare a Solution.

In some embodiments, the ferrum salt may include a mixture of a ferrous salt and a ferric salt at a molar ratio of 2:1. For example, the ferrous salt may be FeSO₄·7H₂O, and the ferrous salt may be FeCl₃·6H₂O. The solvent may include a mixture of absolute ethanol and water, and the volume ratio of water to ethanol may be controlled within a range from 1:1 to 20:1 (e.g., 5:1). A concentration of the ferrum ion in the solution may range from 0.01 mol/L to 1.0 mol/L (e.g., 0.03 mol/L).

### (2) Polyethylene Glycol 6000 May be Added to the Solution.

In some embodiments, a volume ratio of polyethylene glycol 6000 to the solution may range from 1:1 to 1:100 (e.g., 1:25).

### (3) Ammonium Hydroxide (30%) May be Added to the Solution to Adjust a pH Range and Generate Ferroferric Oxide.

In some embodiments, after the solution is in an anaerobic state, ammonium hydroxide may be added to the solution. In some embodiments, pH may be adjusted to 8-10, 9-11, (e.g., 10), etc. In some embodiments, the reaction time may be maintained for a time period, for example, 5 minutes to 15 minutes, 8 minutes to 18 minutes, 10 minutes, etc. In some embodiments, the reaction temperature may range from 20° C. to 90° C. (e.g., 60° C.). In some embodiments, the reaction may be performed under vigorous stirring, and the stirring rate may range from 400 r/h to 1000 r/h (e.g., 800 r/h).

### (4) Attapulgite May be Added to the Solution.

In some embodiments, after the generation of ferroferric oxide, the attapulgite, as a surface shell material, added to may be slowly the solution. The attapulgite may be composited with ferroferric oxide to prepare the magnetic nano adsorbent.

In some embodiments, the attapulgite may be obtained after drying at 120° C. for 4 hours. The attapulgite may be a crystalline hydrated magnesium aluminum silicate mineral, which has a large surface area, high adsorption capacity, ion exchange capacity, and/or special rheological properties. Therefore, by compositing attapulgite with ferroferric oxide, the adsorption and/or hydrophobicity of the magnetic nanoparticles may be enhanced.

In some embodiments, the amount of the attapulgite may be 0.1%-10% (e.g., 1%) of the volume of the solution. In some embodiments, the temperature range of the composition process may range from 40° C.-90° C. (e.g., 80° C.). In some embodiments, the reaction time of the composition process may maintain for 0.5 hours-5 hours (e.g., 2 hours).

In some embodiments, after the composition process is completed, the magnetic particles may be quickly separated under the action of a magnetic field, and washed several times with deionized water to remove unreacted chemical substances until the pH of the solution is around 7. Finally, the black particles are collected and vacuum-dried at a constant temperature. The dried magnetic particles may be used as the magnetic nano adsorbent.

In some embodiments, since Fe₃O₄ is unstable in an aerobic state, the preparation of the magnetic nano adsorbent may be performed in an anaerobic state. Inert gas (e.g., nitrogen gas) may be introduced to remove oxygen. A time period for the oxygen removal may maintain for 30 minutes.

The pH range may include a range from 9 to 11. During the adding the aluminum/ferrum coagulating agent into the fermentation broth to conjugate with the magnetic nano adsorbent to further filter out the second impurity, the pH of the fermentation broth is adjusted to the pH ranging from 4 to 7.

As described above, stage 2 may include: adding the aluminum/ferrum coagulating agent into the filtration solution to conjugate with the magnetic nano adsorbent to further filter out the second impurity.

In some embodiments, the amount of the aluminum/ferrum coagulating agent may be 0.1%-10% (e.g., 0.5%) of the solution volume. In some embodiments, the aluminum/ferrum coagulating agent may include alum (Al₂(SO₄)₃·18H₂O). In some embodiments, the time range of the adsorption process in stage 2 may range from 0.5 hours to 5 hours (e.g., 1 hour). In some embodiments, the temperature of the adsorption process may range from 10° C. to 50° C. (e.g., 25° C.).

In some embodiments, since a zero charge point the magnetic nano-aluminum/ferrum adsorbent may be 7.13, when the pH of the solution in stage 2 is lower than the zero charge point, the surface of the magnetic nano-aluminum/ferrum may be positively attracted to negatively charged proteins, sugars, pigments, or other molecules, and the adsorption may occur through electrostatic interactions. However, when the pH of the solution in stage 2 is larger than the zero charge point, the magnetic nano-aluminum/ferrum adsorbent may be acted as negatively charged particles and as an electrostatic barrier between the adsorbent and the negative surface of the solution, strongly inhibiting the adsorption. Therefore, the pH of the solution in stage 2 may need to be adjusted to be neutral or acidic.

In order to further explore the influence on the adsorption process under different pH, 5 mg of the magnetic nano-aluminum/ferrum adsorbent may be mixed with 2 mL of a protein phosphate buffer solution (pH of 7.13, 0.02 mol/L) with an initial concentration of 1.0 mg/mL, and then adsorption may be performed on a shaking table at 25° C. for 24 h. Since the protein has a maximum absorption peak at 406 nm, the absorbance of the solution before and after adsorption may be measured at 406 nm. The concentration of the protein in the solution may be measured according to a linear relationship between an absorbance range of 0.2-0.8 and a concentration of the protein, by using the following

$$Equation ( 1 )$$

$$Q = ( Ci - Cf ) V / m \qquad ( 1 )$$

where, Q represents the adsorption capacity of the magnetic nano-aluminum/ferrum adsorbent with respect to protein at different pH, mg/g; Ci represents the concentration of the protein solution before the adsorption process, mg/mL; Cf represents the concentration of the protein solution after being adsorbed by the magnetic nano-aluminum/ferrum adsorbent, mg/mL; V represents the volume of protein solution, mL; and m represents the mass of the magnetic nano-aluminum/ferrum adsorbent for adsorbing the protein, g.

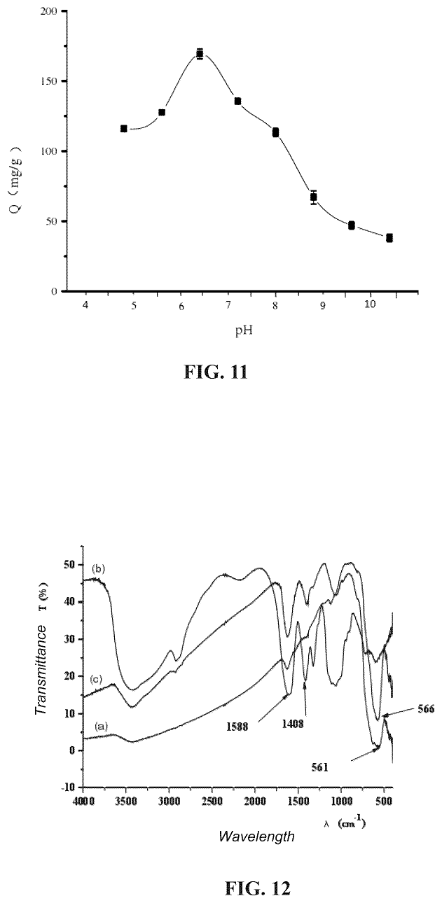

FIG. 11 shows the adsorption result. As shown in FIG. 11, when the pH of the protein solution gradually increases from 4.0 to 6.5, the adsorption amount of the magnetic nano-aluminum/ferrum adsorbent to the protein also gradually increases; when the pH of the protein solution increases from 6.20 to 10, the adsorption amount of the magnetic nano-aluminum/ferrum adsorbent to the protein then gradually decreases. Therefore, the adsorption capacity of the magnetic nano-aluminum/ferrum adsorbent to protein can each the maximum value when the pH of the solution was about 6.5.

In some embodiments, after stage 2, the magnetic nano-aluminum/ferrum adsorbent may be removed by centrifugation or filtration to obtain a clear and/or translucent glyceric acid solution.

In some embodiments, a Fourier transform infrared spectrometer (FTIR) of the Nicolet company of the United States may be used to perform infrared spectrum characterization of the magnetic nano adsorbent of stage 1. A JEM-1200EX (120 KV) transmission scanning electron microscope may be used to characterize the morphology and size of the magnetic nano adsorbent, as well as the removal rate of protein and turbidity.

FIG. 12 shows the result of the FTIR characterization of a coupling reaction. As shown in FIG. 12 (e.g., spectrum 2c), the —OH group of the attapulgite allows the attapulgite to be coupled with Fe₃O₄. In order to further confirm that the attapulgite is successfully attached to the surface of Fe₃O₄, FTIR spectral scanning analysis was performed on pure Fe₃O₄ (e.g., spectrum 2a) and pure attapulgite (e.g., spectrum 2b), and the results are shown in FIG. 12. It can be seen that two characteristic absorption peaks (—NH₂ at 1408 cm⁻¹ and —COOH at 1588 cm⁻¹) of attapulgite appear on the spectrum of the magnetic nano adsorbent, which fully demonstrates that attapulgite has been used successfully to achieve surface modification of Fe₃O₄. In addition, the surface modification of attapulgite can change the infrared spectrum of pure Fe₃O₄, but have little effect on the shift of some characteristic absorption peaks (such as the shift of FeO characteristic absorption peak from 561 cm⁻¹ to 566 cm⁻¹). In addition, no characteristic absorption peak of new functional groups is found in the infrared spectrum of the magnetic nano adsorbent, so it can be concluded that attapulgite has successfully coated and aggregated on Fe₃O₄.

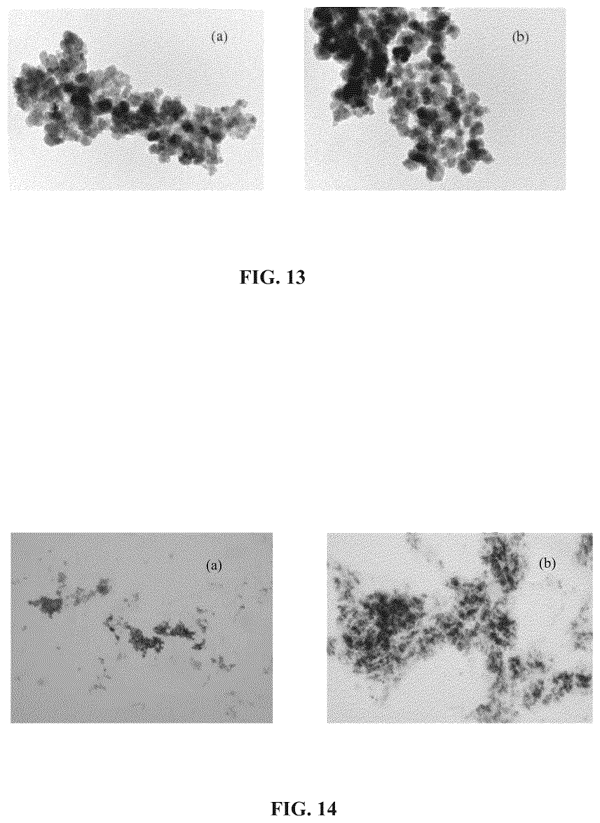

Further, for the particle size characterization of the magnetic nano adsorbent, the magnetic nano adsorbent was ultrasonically dispersed in pure water, and the dispersed solution was dropped on an aluminum foil. After the solvent evaporates, the morphology and dispersion state of the particles can be observed with a suitable magnification. The nanoparticle sizes of pure Fe₃O₄ and the magnetic nano adsorbent were characterized, and the results are shown in FIG. 13(a) and FIG. 13(b), respectively. As shown in FIG. 13(b), the diameter of the magnetic nano adsorbent is about 20 nm. As shown in FIG. 13(a), pure Fe₃O₄ particles are easy to agglomerate through magnetic attraction because there is no wrapping shell on the surface, and there is almost no gap between particles. The magnetic nano adsorbent has functional groups such as Si—O—Si, —COOH, —C═C— and attapulgite on the surface, and the magnetic adsorption and agglomeration phenomenon between particles are reduced. In addition, there are more gaps between particles, the particle distribution is uniform, the dispersion is good, the polymer molecule distribution on the surface is relatively regular, and the thickness of the polymer layer is relatively uniform, such that the particles can achieve good dispersion in aqueous solution. Under the action of an external magnetic field, the magnetic particles can be rapidly aggregated and thus effectively separated from the aqueous phase. After the external magnetic field is withdrawn, the magnetic particles can return to the dispersed state in the solution.

In some embodiments, turbidity may be one of the most important parameters to determine the adsorption effect. The prepared novel adsorbent was compared with a flocculant such as chitosan, activated carbon on the solution obtained in step 1003 under the same condition for adsorption and impurity removal. Protein production, turbidity, and decolorization rate were measured, the results are shown in Table 1.

### Table 1

| | | Protein | Turbidity | | |
|---|---|---|---|---|---|
| | | removal | removal | decolorization | Color of |
| No. | Flocculant | rate % | rate % | ratio % | filtrate |
| 1 | Chitosan | 68.2% | 67.2 | 97.2 | yellowish |
| | | | | | red |
| 2 | Activated carbon | 80.8% | 78.5 | 98.5 | yellow |
| 3 | Prepared magnetic nano | 100% | 93.1 | 90.2 | Faint |
| | adsorbent-aluminum | | | | yellow |
| 4 | Prepared magnetic nano- | 100% | 99.8 | 99.7 | colorless |
| | aluminum/ferrum | | | | |
| | adsorbent | | | | |

As seen from Table 1, impurities such as proteins in the solution cannot be completely removed by using the chitosan or the activated carbon, and the resulting filtrate still has pigment. The magnetic nano adsorbent and/or the magnetic nano-aluminum/ferrum adsorbent prepared in the present disclosure can effectively remove protein, pigment, or other natural organic impurities, and the clear and translucent solution can be obtained.

Compared with the magnetic nano-adsorbent, the turbidity removal rate can be significantly improved by 6.8% after being conjugated with the aluminum/ferrum coagulating agent, and the pigment removal rate can be increased by 9.5%. Therefore, the prepared magnetic nano-aluminum/ferrum adsorbent has good adsorption and flocculation properties.

In 1007, an extraction operation may be performed to filter out a third impurity from the fermentation broth (e.g., the clear and/or translucent solution in 1005) to obtain the purified glyceric acid sample. The third impurity may include glycerin and dihydroxyacetone.

In some embodiments, operation 1007 may include following steps.

(1) organic alcohol may be added into the fermentation broth to allow glyceric acid in the fermentation broth to react with organic alcohol to produce glycerate.

In some embodiments, a volume ratio of the fermentation broth to organic alcohol may be 1:1-1:50 (e.g., 1:5). In some embodiments, organic alcohol may include methanol, ethanol, primary alcohol, secondary alcohol, tertiary alcohol, or the like, or a mixture thereof. For example, organic alcohol may include methanol or ethanol.

In some embodiments, the reaction may be performed under a catalyst. A volume amount of the catalyst may be 0.1%-10% (e.g., 2.5%) of the total feed amount. In some embodiments, the catalyst may include concentrated sulfuric acid, concentrated hydrochloric acid, organic sulfonic acid, sulfonic acid ionic liquid, boric acid, solid superacid catalyst, or the like, or a mixture thereof. For example, the catalyst may include concentrated sulfuric acid.

In some embodiments, a time of the esterification reaction may be controlled within 1 hour-10 hours (e.g., 2.5 hours). The temperature of the esterification reaction may be controlled at 10° C.-90° C. (e.g., 50° C.).

(2) an extraction solution may be formed by extracting glycerate from the fermentation broth using an organic solvent.

In some embodiments, the organic solvent may include a non-polar solvent, for example, petroleum ether, n-hexane, n-pentane, n-heptane, isopentane, cyclohexane, isooctane, tetrachloride, toluene, p-xylene, ether, ethyl acetate, dichloromethane, etc. For example, the organic solvent may include n-hexane. In some embodiments, a volume ratio of the organic solvent to the fermentation broth may be 0.1 times-10 times (e.g., 1). In some embodiments, the extraction process may be carried out by stirring for 10 minutes to 2 hours (e.g., 30 minutes). In some embodiments, the temperature of the extraction process may be controlled at 10° C.-50° C. (e.g., 25° C.).

(3) the extraction solution may be concentrated to obtain a concentrated solution of glycerate.

In some embodiments, the extraction solution may be concentrated at a temperature of 10° C.-50° C. (e.g., 40° C.). In some embodiments, the concentration may finish until there is no solvent separating, that is, glyceride may be highly purified.

(4) the concentrated solution of glycerate may be mixed with water and the pH of the concentrated solution of glycerate may be adjusted to a first pH range to hydrolyze glycerate to produce glyceric acid and organic alcohol; the pH may be adjusted to a second pH range and an extraction solution of glyceric acid may be obtained;

In some embodiments, the first pH range may include a range from 8 to 11, from 10 to 10.5, etc. The second pH range may include a range from 6 to 6.5. In some embodiments, the pH of the concentrated solution of glycerate may be adjusted to the first pH range by adding sodium hydroxide solution (10%). In some embodiments, a volume ratio of glycerate to water may be 1:1-1:10, (e.g., 1:2.5). In some embodiments, the hydrolysis temperature may be controlled at 10° C.-60° C. (e.g., 40° C.). The hydrolysis time may be controlled within 1 hour-20 hours (e.g., 5 hours). In some embodiments, a solvent may be added to obtain the extraction solution of glyceric acid, and the solvent may be the same as the solvent used in (2).

(5) the extraction solution of glyceric acid may be concentrated.

In some embodiments, the extraction solution of glyceric acid may be concentrated under a low-temperature in a vacuum environment. A sample after the concentration may include a high-purified glyceric acid solution without glycerin and dihydroxyacetone. In some embodiments, the concentration condition may be the same as that of the concentration after the esterification and extraction.

Further, 10 batches of experimental verification on the above-mentioned removal of by-products, i.e., glycerin and dihydroxyacetone, may be performed. Ethanol was used for esterification, concentrated sulfuric acid was used as a catalyst, and n-hexane was used as an extraction agent. The purity of the product was measured by high-performance liquid chromatography (HPLC), the conversion rate was calculated, and the results are shown in Table 2.

TABLE 2

| | | Esterification | Purity of | Hydrolysis | Residual | Residual |
|---|---|---|---|---|---|---|
| | Purity of | conversion | glyceric | conversion | amount of | amount of |
| No. | glycerate % | rate % | acid % | rate % | glycerinum % | dioxyacetone % |
| 1 | 99.9 | 98.2 | 100 | 99.3 | — | — |
| 2 | 99.7 | 97.8 | 99.7 | 99.2 | — | 0.03 |
| 3 | 99.9 | 97.9 | 99.9 | 99.2 | — | 0.01 |
| 4 | 99.8 | 98.1 | 99.8 | 99.3 | — | 0.02 |
| 5 | 99.8 | 98.2 | 99.8 | 99.4 | — | 0.02 |
| 6 | 99.9 | 98.1 | 99.9 | 99.2 | — | 0.01 |
| 7 | 99.7 | 98.4 | 99.7 | 99.2 | — | 0.03 |
| 8 | 99.7 | 98.2 | 99.7 | 99.4 | — | 0.03 |
| 9 | 99.8 | 98.3 | 99.8 | 99.5 | — | 0.02 |
| 10 | 99.9 | 98.3 | 100 | 98.5 | — | — |

As indicated by Table 2, it can be seen that the residual by-products, such as glycerin, dihydroxyacetone, in the glyceric acid solution after the removal of impurities such as proteins, pigments, can be effectively removed to obtain a high-purity glyceric acid solution through esterification-extraction in combination with hydrolysis-extraction. The total loss rate is less than 3%, and the purity of obtained glyceric acid exceeds 99.5%.

In some embodiments, after glyceric acid is concentrated, the concentrated solution has a very high viscosity and is not easy to store. In order to ensure the stability and easy storage of glyceric acid, some embodiments may prepare the obtained high-purity glyceric acid into calcium glycerate for utilization and/or storage.

In some embodiments, the preparation process of the calcium glycerate may include the following steps. A certain volume of deionized water may be added to the glyceric acid solution to make the glyceric acid solution reach a certain concentration. In order to make the calcium glycerate exist in the crystalline form, an appropriate amount of anhydrous calcium chloride may be added to the solution and stirred until dissolving, and pH may be adjusted to a slightly acidic to filter. Absolute ethanol with once the volume may be added to the filtrate, stirred evenly, cooled, and crystallized at a low temperature for a time period, and calcium glycerate may be obtained by further filtering, washing, and drying.

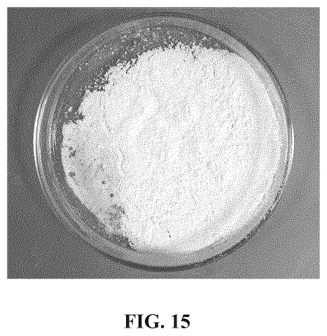

Further, after the cooling crystallization is completed, the solid white filter cake obtained by suction filtration may be separated, the filter cake may be rinsed with absolute ethanol for 2 to 3 times, and finally be put into a vacuum drying oven for vacuum drying to obtain a pure white solid powder (e.g., as shown in FIG. 15).

In some embodiments, after adding the deionized water, the concentration range of the glyceric acid solution may range from 50 g/L-500 g/L (e.g., 250 g/L). In some embodiments, the added amount of the anhydrous calcium chloride may be 1/10-1 times (e.g., 1/2 times) the mass of the glyceric acid solution.

In some embodiments, the temperature when the anhydrous calcium chloride is added and stirred may range from 10° C. to 70° C. (e.g., 25° C.). In some embodiments, the filtration of the calcium glycerate solution may be membrane filtration, and the pore range of the filter membrane may range from 0.1 μm to 5 μm (e.g., 0.22 μm). In some embodiments, the volume ratio of the calcium glycerate solution to ethanol may range from 1:1-1:20 (e.g., 1:2). In some embodiments, the cooling crystallization temperature may range from 0° C. to 20° C. (e.g., 4° C.). The cooling crystallization time may range from 5 hours to 30 hours (e.g., 16 hours).

### Example 5. Preparation of a Magnetic Nano Adsorbent

FeCl₃ 6H₂O and FeSO₄ 7H₂O at a molar ratio of 2:1 were weighted and dissolved in 1 L of an ethanol-water solution (a ratio of ethanol to water being 1:5) under nitrogen protection in a four-neck flask to obtain a sample. The total iron ion concentration was 0.03 mol/L. The sample was stirred and dissolved at a speed of 800 r/h after the dissolution is completed to obtain a dissolved sample. PEG 6000 solution of 25% volume was added to the dissolved sample, the temperature was controlled at 60° C., and nitrogen was introduced to deoxygenate for 30 minutes to obtain a deoxygenated sample. 30% ammonia water (NH₃·H₂O) solution was added to the deoxygenated sample quickly under a mechanical stirring state. When the pH of the solution was adjusted to be larger than 10, the color of the mixture solution immediately changed from orange to black, and a large number of black particles were formed. After the mixture solution reacted and stirred for 10 minutes, 10 g of attapulgite was added to the reacted sample slowly under vigorous stirring, and then the temperature was raised to 80° C. for 2 hours. After the reaction finished to obtain a reacted sample, the magnetic particles were quickly separated from the reacted sampled under the action of an external magnetic field. The separated magnetic particles were washed several times with deionized water to remove unreacted chemical substances until the pH of the final solution was around 7. The black particles were collected and dried in a vacuum oven at 60° C. to obtain the magnetic nano adsorbent.

### Example 6. Preparation of a Fermentation Broth of Glyceric Acid

A solution of Gluconobacter oxydans frozen and preserved in an ultra-low temperature refrigerator at −70° C. was transferred to a plate containing a seed medium. The plate with the solution was placed on a constant temperature shaker at 30° C. with a rotation speed of 200 rpm for 10 hours to cultivate a mature single colony, and then the cultivated primary seed liquid was transferred to a secondary seed medium of a triangular flask with 10% inoculum volume. The liquid volume was 100 ml/1 L. The secondary seed medium with the cultivated primary seed liquid was placed on a shaker for cultivation with a cultivation temperature of 32° C., a rotation speed of 220 rpm, and a cultivation time of 20 hours. When the wet weight of the thallus reached 3 g/L, the thallus was transferred to a seed tank for cultivation.

The cultivation in the seed tank included the following operations. After the pH of the thallus was adjusted to 6, the thallus was sterilized at 115° C. for 30 minutes, and the seed solution in the shake flask was transferred to the seed tank according to 10% inoculum volume. The liquid volume was 100 mL/1 L, the cultivation temperature was 32° C., the tank pressure was 0.05 MPa, and the ventilation ratio was 1.5 VVM, the rotation speed was 500 rpm. During the cultivation process, pH was controlled at about 7.5 by adding ammonia water, and the cultivation period was 24 hours. When the wet weight reached 5 g/L, the seed liquid in the seed tank was moved to a fermenter for cultivation. Components of the seed medium in the fermenter included: peptone 5 g/L, yeast powder 5 g/L, glucose 5 g/L, MgSO₄ 7H₂O 1 g/L, and sodium chloride 5 g/L.

The cultivation in the fermenter (also referred to as fermentation cultivation) included following operations. After pH was adjusted to 7.5, the seed liquid in the seed tank was sterilized at 121° C. for 20 minutes and transferred from the seed tank to a 20 L fermenter according to 10% inoculum volume. The liquid volume was 15 L, and initial culture conditions included a cultivation temperature of 32° C., a rotational speed of 200 rpm, an aeration ratio of 0.5 VVM, and a tank pressure of 0.03 MPa. During the culture process, pH was controlled at about 7.5 by supplementing ammonia water, and the cultivation time was 108 h. During the cultivation process, the feeding concentration was 500 g/L glucose solution and 2 g/L ammonium sulfate. Components of the fermentation medium included: glycerol 150 g/L, KH2PO4 0.9 g/L, K2HPO4 0.1 g/L, zinc chloride 0.015 g/L, MgSO4·7H2O 1 g/L, peptone 9 g/L, and yeast powder 1 g/L.

When the growth rate of the product obviously slowed down or the thallus was shallow, the fermentation was stopped. The glyceric acid content in the fermentation broth was measured by HPLC. The glyceric acid content can reach 100 g/L when the fermentation was terminated.

### Example 7: Preparation of a Purified Calcium Glycerate Sample Using a Magnetic Nano-Aluminum/Ferrum Adsorbent

(1) 1 L of a glyceric acid fermentation broth was prepared in Example 6, and bacteria were removed through a 0.22 μm ceramic membrane at 0.3 MPa to obtain 970 mL of a clear solution of glyceric acid.

(2) pH of the clear solution of glyceric acid was adjusted to 4.8, adding 19.4 g of a magnetic nano adsorbent solution with a volume of 2% was added to the clear solution at 25° C. and stirred at a speed of 80 r/h. After absorption for 2 hours, 4.85 g of alum solution with a volume of 0.5% was added and the stirring was continued for 1 hour to complete the flocculation for impurity removal, and the reacted solution was stood still and the flocculation situation was observed. Under the flocculation situation, the volume of the flocs produced was large (see FIG. 14(b)), the sedimentation speed was fast, and the clear solution of the flocculation turned colorless. The clear solution of the flocculation was sampled and measured to determine that the removal rate of protein in the flocculation solution was 100%, the removal rate of turbidity was 99.5%, the purity of glyceric acid was 95.3%, and the retention rate of active ingredients of glyceric acid was 98.8%.

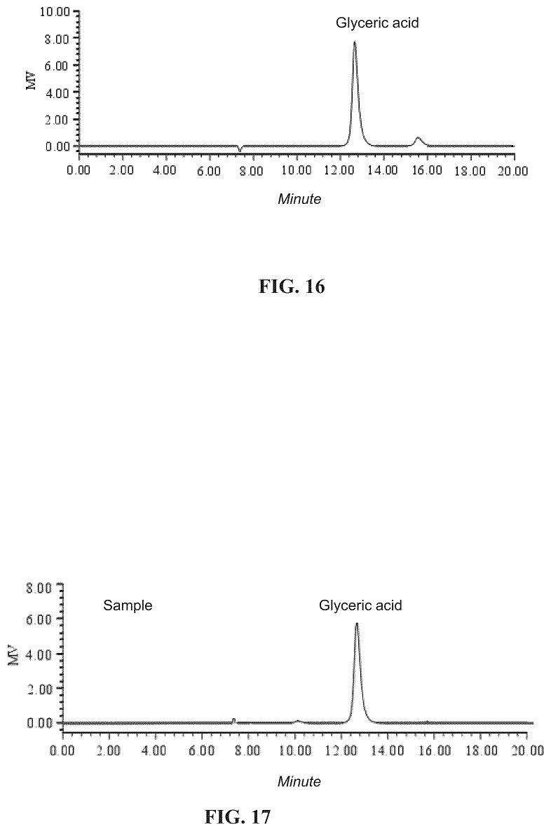

(3) Ethanol solution with 5 times the volume was added to 960 ml of the clear solution of glyceric acid after impurity removal, and then 24 ml of concentrated sulfuric acid with a volume being 2.5% of the glyceric acid solution was added to the solution. The temperature was raised to 50° C. under which the solution is stirred for 2.5 hours. After the reaction is completed, n-hexane with once the volume of the reaction solution was added to the reaction solution, and the stirring and extracting were performed. The upper organic phase was separated after extraction for 30 minutes. The upper organic phase was dried under vacuum at 40° C. to obtain 105 g of glyceride ester solution. Deionized water with 2.5 times the weight was added to the obtained glyceride solution and stirred evenly. The temperature was controlled at 40° C., and pH was adjusted to 10.5 by 10% sodium hydroxide solution to carry out the hydrolysis reaction. The hydrolysis reaction may be maintained for 5 hours. N-hexane with once the volume was added to the reaction solution, and the stirring was performed for 30 minutes for extraction. The upper organic phase was separated, and dried under vacuum at 40° C. to obtain 104 g of glyceric acid concentration solution. The purity of the obtained glyceric acid solution was 99.1% (see the purity spectrum in FIG. 16), and the loss rate was 2.35%.

(4) The purified glyceric acid concentration solution was diluted to a concentration of 250 g/L, anhydrous calcium chloride (with ½ times the mass of glyceric acid) was added to the solution, and the stirring was performed until dissolving. pH was adjusted to 6.7, and the dissolved solution was filtered through a 0.22 μm filter membrane to obtain a calcium glycerate solution. Absolute ethanol (with twice the volume) was added to the calcium glycerate solution and stirred for 30 minutes, and then the solution was input in a refrigerator at 4° C. for cooling and crystallizing for 16 hours. After the crystallization was completed, suction filtration was performed to separate and obtain a solid white filter cake. Absolute ethanol was used for washing the filter cake 2-3 times.

(5) The washed crystal was input into a drying oven at 45° C. and dried in a vacuum to a constant weight to obtain a fine calcium glycerate product with a weight of 102.7 g, content of 99.15%, purity of 99.72%, no detected protein content, the crystallization yield of 97.92%, the total yield: 95.57%. The obtained fine calcium glycerate was a pure white powder (see FIG. 15), pure color, fine particles, good dispersion, loose bulk density of 0.53 g/ml (crystal yield=crystal weight*crystal content/(concentrate content*concentrate volume)*100, total yield=crystallization yield*retention rate after decolorization/100);

The calcium glycerate product (purity of 99.9%) of SIGMA-ALDRICH company was selected as the standard product. An HPLC analysis was performed on the fine calcium glycerate sample prepared above, and the result is shown in FIG. 17. A Fourier transform infrared spectroscopic analysis was performed on the fine calcium glycerate sample prepared above, and the results are shown in FIG. 8.

As shown in FIG. 17, the peak time of the standard product and the prepared calcium glycerate sample are consistent, both at about 12.66 min, which can be identified as the same substance, and only a small miscellaneous peak (for the homologue dihydroxyacetone) appears in the sample spectrum, indicating that the sample contains few impurities, and the purity of the refined product obtained through integral calculation is 99.1%.

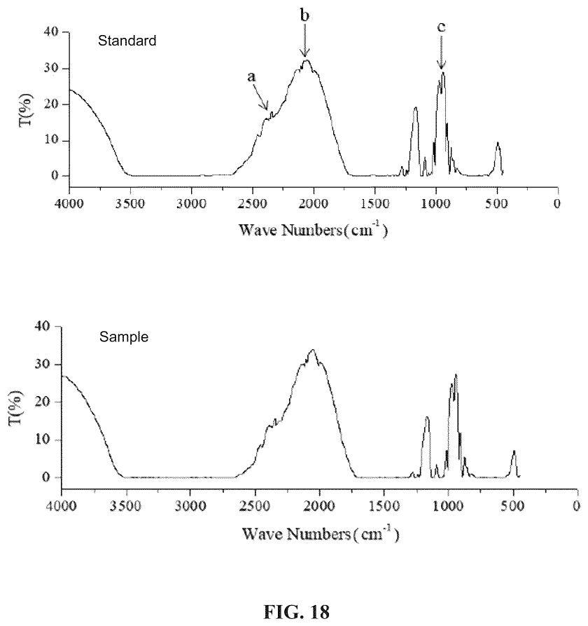

As shown in FIG. 18, the absorption peak of —OH is at 2050-2550 (characteristic peak a), the absorption peak of C═O appears at 1720-2050 (characteristic peak b), and carboxylate compounds generally produce a band with a large intensity change (characteristic peak c) at 800-1200. It can be seen from FIG. 18 that the peak position of the main functional group is basically the same as that of the standard product, which can be identified as the same substance.

### Example 8: Preparation of a Purified Calcium Glycerate Sample Using Chitosan

(1) 10 L of a glyceric acid fermentation broth prepared in Example 6, was weighted and bacteria through a 0.22 μm ceramic membrane at 0.3 MPa were removed to obtain 9.7 L of a clear solution of glyceric acid;
(2) The temperature of the clear solution of glyceric acid was controlled at 25° C., and 3.88 g of chitosan was added to the solution. The stirring was performed at a speed of 5 r/min for 43 minutes. After the stirring was stopped, the solution stood still and the flocculation condition was observed. The color of the flocculated solution was yellow. The floc particles were small (see FIG. 14 (a)), and the sedimentation rate was slow. The removal rate of protein in the glyceric acid solution was 35.8%, and the removal rate of turbidity was 69.5%. The obtained glyceric acid solution was yellow-red, the retention rate of the active ingredient of glyceric acid was 90.8%, and the purity of glyceric acid was 75.3%;
(3) Ethanol solution with 5 times the volume was added to 950 ml of the glyceric acid solution after impurity removal, and then 24 ml of concentrated sulfuric acid with a volume being 2.5% of the glyceric acid solution was added. The temperature was raised to 50° C. and the stirring was performed for 2.5 h. After the reaction was completed, n-hexane (with once the volume of the reaction solution) was added, and the stirring and extracting were performed. The upper organic phase was separated after extraction for 30 minutes and dried at 40° C. to obtain 103 g of a glyceride ester solution. Deionized water (with 2.5 times the weight of the obtained glyceride solution) was added to the obtained glyceride solution and stirred evenly. The temperature was controlled at 40° C., pH was adjusted to 10.5 by 10% sodium hydroxide solution to perform the hydrolysis reaction, and the hydrolysis was completed after 5 hours of reaction. N-hexane (once the volume) was added, and the stirring and extracting were performed for 30 minutes. The upper organic phase was separated and dried under vacuum at 40° C. to obtain 102 g of a glyceric acid concentration solution. The purity of the obtained glyceric acid concentration solution was 88.6%, the conversion lost was 2.1%, and the color was dark brown.
(4) The glyceric acid concentration solution was diluted to a concentration of 250 g/L, anhydrous calcium chloride (with 1/2 times the mass of glyceric acid) was added and stirred until dissolving. After the pH was adjusted to 6.7, the solution was filtered through a 0.22 μm filter membrane to obtain a calcium glycerate solution. Absolute ethanol with twice the volume was added to the calcium glycerate solution and stirred for 30 minutes, and then the solution was placed in a refrigerator at 4° C. for cooling and crystallizing for 16 hours. After the crystallization was completed, suction filtration was performed to separate and obtain a solid white filter cake, and the filter cake was washed with absolute ethanol 2-3 times;
(5) The crystal after washing was input into a drying oven at 45° C. and dried in a vacuum to a constant weight to obtain a calcium glycerate sample with a weight of 109 g, a content of 90.51%, protein content of 0.21%, a crystallization yield of 96.72%, and a total yield of 86.06%. The obtained fine calcium glycerate sample was light brown powder, and the loose bulk density was 0.43 g/ml (crystal yield=crystal weight*crystal content/(concentrate content*concentrate volume)*100, total yield=crystallization yield*retention rate after decolorization/100).

### Example 9: Preparation of a Purified Calcium Glycerate Sample Using a Magnetic Nano Adsorbent

(1) 1 L of a glyceric acid fermentation solution prepared in Example 6 was weighted, and bacteria were removed through a 0.22 μm ceramic membrane at 0.3 MPa to obtain 970 mL of a clear solution of glyceric acid.

(2) the pH of the clear solution of glyceric acid was adjusted to 4.8, and 19.4 g of the magnetic nano adsorbent with a volume being 2% of the solution was added at 25° C. and stirred at a speed of 80 r/h. After absorption for 2 hours, the solution was filtered. The filtration situation was observed, and the filtrate was pale yellow. The removal rate of protein in the filtrate was 100%, the removal rate of turbidity was 93.5%, the purity of glyceric acid was 90.3%, and the retention rate of active ingredients of glyceric acid was 99.1%.

(3) Ethanol solution with 5 times the volume was added to 960 ml of the clear solution of glyceric acid after impurity removal, and then 24 ml of concentrated sulfuric acid with a volume being 2.5% of the glyceric acid solution was added. The temperature was raised to 50° C. and stirred for 2.5 hours. After the reaction was completed, n-hexane with once (i.e., 1 time) the volume of the reaction solution was added, and the stirring and extracting were performed. The upper organic phase was separated after extraction for 30 minutes and dried under vacuum at 40° C. to obtain 105 g of glyceride ester solution. Deionized water with 2.5 times the weight was added to the obtained glyceride solution and stirred evenly. The temperature was controlled at 40° C., and pH was adjusted to 10.5 by 10% sodium hydroxide solution to carry out the hydrolysis reaction. The hydrolysis reaction was completed after 5 hours. N-hexane with once the volume was added, and stirred for 30 minutes. Then, the extracting was performed. The upper organic phase was separated and dried under vacuum at 40° C. to obtain 104 g of glyceric acid concentration solution. The purity of the obtained glyceric acid solution was 98.6%, and the loss rate was 2.05%;

(4) The purified glyceric acid concentration solution was diluted to a concentration of 250 g/L, and anhydrous calcium chloride (with 1/2 times the mass of glyceric acid) was added and stirred until dissolving. pH was adjusted to 6.7, and the solution was filtered through a 0.22 μm filter membrane to obtain a calcium glycerate solution. Absolute ethanol (with twice the volume) was added to the calcium glycerate solution and stirred for 30 minutes, and then the solution was input in a refrigerator at 4° C. for cooling and crystallizing for 16 hours. After the crystallization was completed, suction filtration was performed to separate and a solid white filter cake was obtained. The solid white filter cake was washed using absolute ethanol 2-3 times;

(5) The washed crystal was put into a drying oven at 45° C. and dried in a vacuum to a constant weight to obtain a fine calcium glycerate product with a weight of 103.5 g, a purity of 97.58%, no detected protein content, a crystallization yield of 97.14%, a total yield of 94.29%. The obtained fine calcium glycerate was a milky white powder, and loose bulk density was 0.39 g/ml (crystal yield=crystal weight*crystal content/(concentrate content*concentrate volume)*100, total yield=crystallization yield*retention rate after decolorization/100);

According to some embodiments of the present disclosure, a composition may be provided. The composition may include a glycerate substance and an excipient. The glycerate substance may include D-glyceric acid or a salt of D-glyceric acid. In some embodiments, the glycerate substance may include the salt of D-glyceric acid of crystalline Forms I, II, or amorphous III, the D-glyceric acid or the salt of D-glyceric acid purified from a fermentation broth as described above, etc. In some embodiments, the composition may promote the metabolic process of alcohol, thereby protecting the liver.

In some embodiments, a mass ratio of the glycerate substance to the excipient may be (0.01-300):1. For example, the mass ratio of the glycerate substance to the excipient may be 0.01:1, 1:1, 5:1, 10:1, 20:1, 30:1, 40:1, 50:1, 60:1, 70:1, 80:1, 90:1, 100:1, 120:1, 150:1, 180:1, 200:1, 220:1, 250:1, 280:1, 300:1, etc.

In some embodiments, the excipient may include at least one of an NAD+ precursor, a mineral, or a vitamin.

The metabolic process of alcohol may be that ethanol is converted into acetaldehyde under the action of alcohol dehydrogenase (ADH), acetaldehyde is transferred to the mitochondria of liver cells and converted into acetic acid under the action of acetaldehyde dehydrogenase (ALDH) in the mitochondria, and acetic acid is finally converted into carbon dioxide and water. The process of converting ethanol into acetaldehyde and acetaldehyde into acetic acid may require the participation of NAD+, and NAD+ is converted into NADH (nicotinamide adenine dinucleotide) during the process. The supplementation of an NAD+ precursor can increase the amount of NAD+ in the body, which can accelerate the metabolic process of ethanol and acetaldehyde in the body. The supplement of the mineral and/or the vitamin can increase the activity of ADH and ALDH.

In some embodiments, in order to accelerate the metabolic process of alcohol, the excipient may include two of the NAD+ precursor, the mineral, and the vitamin. For example, the excipient may include the NAD+ precursor and the mineral, the NAD+ precursor and the vitamin, or the mineral and the vitamin.

In some embodiments, in order to accelerate the metabolic process of alcohol, the excipient may include a mixture of the NAD+ precursor, the mineral, and the vitamin. A mass ratio of the glycerate substance, the NAD+ precursor, the mineral, and the vitamin may be 100:(0.01-100):(0.01-100):(0.01-100). For example, the mass ratio of the glycerate substance and one of the NAD+ precursor, the mineral, and the vitamin may be 100:0.01, 100:1, 100:5, 100:10, 100:20, 100:30, 100:40, 100:50, 100:60, 100:70, 100:80, 100:90, 100:100, etc.

In some embodiments, the NAD+ precursor may include nicotinamide ribose (NR), nicotinamide mononucleotide (NMN), nicotinic acid (NA), tryptophan (Trp), nicotinamide, or the like, or any mixture thereof. NR may act as a coenzyme for transferring hydrogen ions and play an important role in alcohol metabolism, and the supplementation of NR may increase the level of NAD+ in the blood. NMN may be a key intermediate substance for the synthesis of NAD+. NA, as the vitamin B group, may be converted into nicosamide which is a component of coenzyme I and coenzyme II. Nicosamide of the two coenzymes may have reversible hydrogenation and dehydrogenation characteristics, thereby playing a role in the transfer of hydrogen. Trp may be converted into niacin after oxidation, and be a precursor for the synthesis of NAD and NADP. Niacinamide may be an amide compound of nicotinic acid and a component of coenzyme I and coenzyme II.

In some embodiments, in order to accelerate the metabolic process of alcohol, the NAD+ precursor may include a mixture of NR, NMN, NA, Trp, and nicotinamide. A mass ratio of NR, NMN, NA, Trp, and nicotinamide may be (1-3):(1-3):(0.5-2):(0.5-2):1. For example, based on the content of nicotinamide as 1 part by weight, the contents of NR and NMN may each be 1, 1.2, 1.5, 1.8, 2, 2.2, 2.5, 2.8, 3 parts by weight or any value therebetween. The contents of NA and Trp may each be 0.5, 0.8, 1, 1.2, 1.5, 1.8, 2 parts by weight or any value therebetween.

In some embodiments, the mineral may include a calcium substance, a magnesium substance, a zinc substance, a selenium substance, or the like, or any mixture thereof. Since both ADH and ALDH are zinc-containing metalloenzymes, the supplementation of the zinc substance may be beneficial to the improvement of the activity of ADH and ALDH. Calcium element may be a regulator of the citric acid cycle, and the use of the calcium substance may be beneficial to the catabolism of acetic acid. In some embodiments, in order to accelerate the metabolic process of alcohol, the mineral may include a mixture of the calcium substance, the zinc substance, and the magnesium substance. In some embodiments, in order to accelerate the metabolic process of alcohol, the mineral may include a mixture of the calcium substance, the zinc substance, and the selenium substance.

In some embodiments, the calcium substance may include calcium lactate, calcium citrate, calcium carbonate or calcium gluconate, or the like, or a mixture thereof. The magnesium substance may include magnesium oxide, magnesium lactate, magnesium citrate, magnesium amino acid, magnesium gluconate, or the like, or a mixture thereof. The zinc substance may include zinc lactate, zinc chloride, zinc sulfate, zinc gluconate, or the like, or a mixture thereof. The selenium substance may include sodium selenite, selenium-enriched yeast, or the like, or a mixture thereof.

In some embodiments, in order to accelerate the metabolic process of alcohol, the mineral may include a mixture of the calcium substance, the magnesium substance, the zinc substance, and the selenium substance. A mass ratio of the calcium substance, the magnesium substance, the zinc substance, and the selenium substance may be (0.5-2):(0.5-2):(0.5-2):1. For example, based on the content of the selenium substance as 1 part by weight, the contents of the calcium substance, the magnesium substance, the zinc substance may each be 0.5, 0.8, 1, 1.2, 1.5, 1.8, 2 parts by weight or any value therebetween.

In some embodiments, the vitamin may include vitamin B, vitamin C, or the like, or a mixture thereof. Vitamin B, as a component of coenzymes or enzyme proteins, can promote the biological activities of coenzymes ADH and ALDH, and also have a certain enhancement effect on the ability of the liver to metabolize alcohol. Vitamin C may have the effect of anti-oxidation and anti-free radicals, and can alleviate the situation of free radicals caused by drinking alcohol.

In some embodiments, in order to accelerate the metabolic process of alcohol, the vitamin may include a mixture of vitamin B1, vitamin B2, vitamin B6, and vitamin C. A mass ratio of vitamin B1, vitamin B2, vitamin B6, and vitamin C may be (0.5-2):(0.5-2):(0.5-2):1. For example, based on the content of vitamin C as 1 part by weight, the contents of vitamin B1, vitamin B2, and vitamin B6 may each be 0.5, 0.8, 1, 1.2, 1.5, 1.8, 2 parts by weight or any value therebetween.

In some embodiments, the preparation method of the composition may include mixing the glycerate substance and the excipient uniformly. Steps such as crushing, sieving, and drying may also be performed before the mixture. For example, the sieve size may include 50-200 mesh, 60-100 mesh, etc.

In some embodiments, the composition may be utilized in a form of a soft capsule, a hard capsule, a microcapsule, a tablet, a powder, a pill, an emulsion, or a suspension.

The present disclosure may further illustrate the composition in detail below through examples.

### 1. Preparation

In the examples, the content of the glycerate substance (e.g., D-glyceric acid, D-glycerate) was measured by high-performance liquid chromatography (HPLC). A detector of the HPLC includes a differential detector. A chromatographic column of the HPLC was shodex SH1011, and the column temperature was 45° C. The mobile phase of the HPLC was 5 mM H₂SO₄, and the flow rate was 1.0 mL/min. The injection volume was 20 μL.

Preparation Examples 10-31 and comparative examples 1-8 were obtained by mixing the glycerate substance and the excipient uniformly at room temperature. The excipient was selected from at least one of an NAD+ precursor, a mineral, or a vitamin. The formulations of the examples are shown in Table 3 below.

TABLE 3

| | Glycerate | NAD+ | Mineral | Vitamin |
|---|---|---|---|---|
| | substance | precursor | (Weight | (Weight |
| Example | (Weight part) | (Weight part) | part) | part) |
| Preparation example | 12 | 15 | / | / |
| 10 | | | | |
| Preparation example | 12 | / | 15 | 1 |
| 11 | | | | |
| Preparation example | 12 | / | / | 15 |
| 12 | | | | |
| Preparation example | 12 | 15 | 15 | / |
| 13 | | | | |
| Preparation example | 12 | 15 | / | 15 |
| 14 | | | | |
| Preparation example | 12 | / | 15 | 15 |
| 15 | | | | |
| Preparation example | 10 | 10 | 10 | 10 |
| 16 | | | | |
| Preparation example | 10 | 15 | 15 | 15 |
| 17 | | | | |
| Preparation example | 10 | 20 | 20 | 20 |
| 18 | | | | |
| Preparation example | 12 | 10 | 10 | 10 |
| 19 | | | | |
| Preparation example | 12 | 15 | 15 | 15 |
| 20 | | | | |
| Preparation example | 12 | 20 | 20 | 20 |
| 21 | | | | |
| Preparation example | 15 | 10 | 10 | 10 |
| 22 | | | | |
| Preparation example | 15 | 15 | 15 | 15 |
| 23 | | | | |
| Preparation example | 10 | 10 | 10 | 10 |
| 24 | | | | |
| Preparation example | 10 | 10 | 10 | 10 |
| 25 | | | | |
| Preparation example | 10 | 10 | 10 | 10 |
| 26 | | | | |
| Preparation example | 10 | 10 | 10 | 10 |
| 27 | | | | |
| Preparation example | 10 | 10 | 10 | 10 |
| 28 | | | | |
| Preparation example | 10 | 10 | 10 | 10 |
| 29 | | | | |
| Preparation example | 10 | 10 | 10 | 10 |
| 30 | | | | |
| Preparation example | 10 | 10 | 10 | 10 |
| 31 | | | | |
| Comparative | 12 | / | / | / |
| example 1 | | | | |
| Comparative | / | 15 | / | / |
| example 2 | | | | |
| Comparative | / | / | 15 | / |
| example 3 | | | | |
| Comparative | / | / | / | 15 |
| example 4 | | | | |
| Comparative | 27 | / | / | / |
| example 5 | | | | |
| Comparative | / | 27 | / | / |
| example 6 | | | | |
| Comparative | / | / | 27 | / |
| example 7 | | | | |
| Comparative | / | / | / | 27 |
| example 8 | | | | |

With respect to preparation examples 10-31 an comparative examples 1-8, the glycerate substance included D-glyceric acid. The excipient included a mixture of an NAD+ precursor, a mineral, and a vitamin. As used herein, the NAD+ precursor included a mixture of NR, NMN, NA, Trp, and nicotinamide of a mass ratio of 2:2:1:1:1, and the mineral included calcium gluconate, magnesium oxide, zinc lactate, and sodium selenite of a mass ratio of 1:1:1:1. The vitamin included a mixture of V_{B1} V_{B2}, V_{B6}, and V_{C} of a mass ratio of 1:1:1:1.

With respect to preparation example 24, the glycerate substance included sodium D-glycerate. The excipient included a mixture of an NAD+ precursor, a mineral, and a vitamin. As used herein, the NAD+ precursor included NR, and the mineral included calcium gluconate. The vitamin included V_{B1}.

With respect to preparation example 25, the glycerate substance included calcium D-glycerate. The excipient included a mixture of an NAD+ precursor, a mineral, and a vitamin. As used herein, the NAD+ precursor included NMN, the mineral may include zinc lactate, and the vitamin included V_{B2}.

With respect to preparation Example 26, the glycerate substance included a mixture of D-glyceric acid and sodium D-glycerate of a mass ratio of 1:1. The excipient included a mixture of an NAD+ precursor, a mineral, and a vitamin. As used herein, the NAD+ precursor included nicotinic acid (NA), the mineral included magnesium oxide, and the vitamin included V_{B6}.

With respect to preparation example 27, the glycerate substance included sodium glycerate. The excipient included a mixture of an NAD+ precursor, a mineral, and a vitamin. As used herein, the NAD+ precursor included tryptophan (Trp), the mineral included sodium selenite, and the vitamin included V_{B2}.

With respect to preparation example 28, the glycerate substance included sodium D-glycerate. The excipient included a mixture of an NAD+ precursor, a mineral, and a vitamin. As used herein, the NAD+ precursor included nicotinamide, the mineral included zinc lactate, and the vitamin included V_{C}.

With respect to preparation example 29, the glycerate substance included D-glyceric acid. The excipient included a mixture of an NAD+ precursor, a mineral, and a vitamin. As used herein, the NAD+ precursor included a mixture of NR and NMN of a mass ratio of 1:1, the mineral included a mixture of calcium gluconate and zinc lactate of a mass ratio of 1:1, and the vitamin included a mixture of V_{B1} and V_{C} of a mass ratio of 1:1.

With respect to preparation example 30, the glycerate substance included D-glyceric acid. The excipient included a mixture of an NAD+ precursor, a mineral, and a vitamin. As used herein, the NAD+ precursor included a mixture of NR and tryptophan of a mass ratio of 1:1, the mineral included a mixture of calcium gluconate and magnesium oxide of a mass ratio of 1:1, the vitamin included a mixture of V_{B2}:V_{C} of a mass ratio of 1:1.

With respect to preparation example 31, the glycerate substance included D-glyceric acid. The excipient included a mixture of an NAD+ precursor, a mineral, and a vitamin. As used herein, the NAD+ precursor included NR, the mineral included sodium selenite, and the vitamin included V_{E}.

### 2. Alcohol Metabolism Test

The test was divided into test groups and comparative groups. Each group included 7 healthy animals (e.g., mice). The healthy animals were fed in the laboratory for 10 days to adapt to the test environment, and then fasting was maintained 12 hours before the start of the test.

4.5 g of the compositions obtained in preparation examples 10-31 and comparative examples 1-8 were respectively dissolved in 15 mL of physiological saline to prepare composition solutions. The animals in the test groups were fed with 2 mL of the composition solution respectively, and the animals in the comparative group were fed with the same dose of normal saline. 1 hour later, the animals in the test groups and the comparative groups were fed with 2 mL of 5% edible ethanol, respectively. 1 hour later, the venous blood of the animals was collected. The ethanol content in the venous blood was detected by a gas chromatography-hydrogen flame ionization detector. The result is shown in Table 4 below.

TABLE 4

| | | Ethanol | Percentage decrease |
|---|---|---|---|
| | | content | compared to |
| Group | Diet | (mg/100 mL) | comparative group |
| 1 | Comparative group | 75.0 | — |
| | (physiological saline) | | |
| 2 | Preparation example | 60.3 | 19.6% |
| | 10 | | |
| 3 | Preparation example | 62.4 | 16.8% |
| | 11 | | |
| 4 | Preparation example | 63.1 | 15.9% |
| | 12 | | |
| 5 | Preparation example | 57.4 | 23.5% |
| | 13 | | |
| 6 | Preparation example | 59.3 | 20.9% |
| | 14 | | |
| 7 | Preparation example | 58.9 | 21.5% |
| | 15 | | |
| 8 | Preparation example | 34.0 | 54.7% |
| | 16 | | |
| 9 | Preparation example | 37.2 | 50.4% |
| | 17 | | |
| 10 | Preparation example | 41.6 | 44.5% |
| | 18 | | |
| 11 | Preparation example | 36.1 | 51.9% |
| | 19 | | |
| 12 | Preparation example | 37.0 | 50.7% |
| | 20 | | |
| 13 | Preparation example | 42.3 | 43.6% |
| | 21 | | |
| 14 | Preparation example | 36.9 | 50.8% |
| | 22 | | |
| 15 | Preparation example | 40.1 | 46.5% |
| | 23 | | |
| 16 | Preparation example | 51.3 | 31.6% |
| | 24 | | |
| 17 | Preparation example | 52.2 | 30.4% |
| | 25 | | |
| 18 | Preparation example | 48.1 | 35.9% |
| | 26 | | |
| 19 | Preparation example | 52.9 | 29.5% |
| | 27 | | |
| 20 | Preparation example | 52.5 | 30.0% |
| | 28 | | |
| 21 | Preparation example | 48.9 | 34.8% |
| | 29 | | |
| 22 | Preparation example | 49.3 | 34.3% |
| | 30 | | |
| 23 | Preparation example | 53.6 | 28.5% |
| | 31 | | |
| 24 | Comparative example | 63.9 | 14.8% |
| | 1 | | |
| 25 | Comparative example | 70.8 | 5.6% |
| | 2 | | |
| 26 | Comparative example | 73.1 | 2.5% |
| | 3 | | |
| 27 | Comparative example | 72.4 | 3.5% |
| | 4 | | |
| 28 | Comparative example | 63.6 | 15.2% |
| | 5 | | |
| 29 | Comparative example | 70.0 | 6.7% |
| | 6 | | |
| 30 | Comparative example | 72.6 | 3.2% |
| | 7 | | |
| 31 | Comparative example | 72.0 | 4.0% |
| | 8 | | |

As indicated by Table 4, when the composition includes a mixture of the glycerate substance, the NAD+ precursor, the mineral, and/or the vitamin, the percentage decrease of ethanol is larger than when the composition includes only one of the glycerate substance, the NAD+ precursor, the mineral, and/or the vitamin.

As further indicated by Table 4, when at least one of the glycerate substance, the NAD+ precursor, the mineral, and/or the vitamin is a mixture, the percentage decrease of ethanol is larger than when each of the glycerate substance, the NAD+ precursor, the mineral, and/or the vitamin includes a single substance. When only one of the glycerate substance, the NAD+ precursor, the mineral, and/or the vitamin is a mixture, the percentage decrease of ethanol is non-obvious. When two or three of the glycerate substance, the NAD+ precursor, the mineral, and/or the vitamin are mixtures, the percentage decrease of ethanol was relatively improved. When all of the glycerate substance, the NAD+ precursor, the mineral, and/or the vitamin are mixtures, the percentage decrease of ethanol is significantly improved.

It should be noted that the above description of the basic concepts is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A crystalline form of calcium D-glycerate of formula (I) 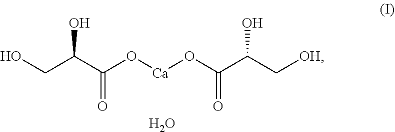
wherein the crystalline form is characterized by having X-ray powder diffraction pattern (XRPD) diffraction peaks (2θ degrees) at 12.9°±0.2°, 20.9°±0.2°, and 31.7°±0.2°.

2. The crystalline form of claim 1, wherein the crystalline form is further characterized by having XRPD diffraction peaks (2θ degrees) at 25.4°±0.2° and 26.1°±0.2°.

3. The crystalline form of claim 1, wherein the crystalline form is further characterized by having XRPD diffraction peaks (2θ degrees) at 15.6°±0.2°, 16.6°±0.2°, 18.8°±0.2°, 22.3°±0.2°, 23.1°±0.2°, 27.4°±0.2°, 27.8°±0.2°, 33.3°±0.2°, 35.2°±0.2°, and 38.2°±0.2°.

4. The crystalline form of claim 1, wherein the crystalline form is prepared by:
mixing D-glyceric acid with a calcium salt to form a first solution;
stirring the first solution at a stirring rate and cooling the first solution to a temperature at a cooling rate;
aging the first solution for a time period at the temperature;
filtering the first solution to obtain a first calcium glycerate sample and drying the first calcium glycerate sample;
redissolving the dried first calcium glycerate sample to form a second solution at a second temperature for a second time period; and
filtering the second solution to obtain a second calcium glycerate sample and drying the second calcium glycerate sample to obtain the crystalline form of calcium glycerate.

5. The crystalline form of claim 4, wherein
the stirring rate ranges from 10 r/min to 40 r/min or from 20 r/min to 25 r/min,
the cooling rate ranges from 3° C./h to 8° C./h or from 4° C./h to 6° C./h,
the temperature ranges from 3° C. to 20° C. or from 5° C. to 15° C., or
the time period ranges from 0.25 h to 8 h or from 2 h to 4 h.

6. The crystalline form of claim 1, wherein the crystalline form is prepared by:
mixing D-glyceric acid with a calcium salt to form a first solution;
adding a solvent into the first solution and stirring at a stirring rate to form a second solution;
aging the second solution for a time period;
filtering the second solution to obtain a first calcium glycerate sample and drying the first calcium glycerate sample;
redissolving the dried first calcium glycerate sample to form a third solution at a second temperature for a second time period; and
filtering the third solution to obtain a second calcium glycerate sample and drying the second calcium glycerate sample to obtain the crystalline form of calcium glycerate.

7. The crystalline form of claim 6, wherein
the solvent includes at least one of methanol, ethanol, isopropanol, glycerol, 1,2-propanediol, 1,3-propanediol, or ethylene glycol,
a volume ratio of the solvent to the first solution ranges from 1/6 to 3 or from 1/3 to 1,
the stirring rate ranges 10 r/min to 40 r/min or from 25 r/min to 35 r/min, or
the time period ranges from 0.25 h to 4 h or from 1 h to 2 h.

8-9. (canceled)

10. The crystalline form of claim 4, wherein
the D-glyceric acid and the calcium salt are mixed at a temperature ranging from 20° C. to 60° C. or from 30° C. to 50° C.,
the second temperature ranges from 20° C. to 60° C. or from 35° C. to 50° C., or
the second time period ranges from 0.2 h to 2 h or from 0.5 h to 1 h.

11-12. (canceled)

13. The crystalline form of claim 6, wherein
the D-glyceric acid and the calcium salt are mixed at a temperature ranging from 20° C. to 60° C. or from 30° C. to 50° C.,
the second temperature ranges from 20° C. to 60° C. or from 35° C. to 50° C., or
the second time period ranges from 0.2 h to 2 h or from 0.5 h to 1 h.

14. A crystalline form of calcium D-glycerate of formula (II) 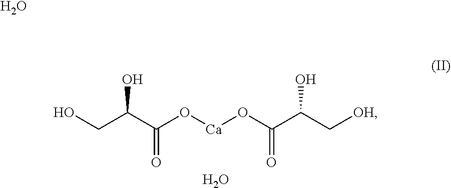
wherein the crystalline form is characterized by having X-ray powder diffraction pattern (XRPD) diffraction peaks (2θ degrees) at 15.4°±0.2°, 17.0°±0.2°, and 27.1°±0.2°.

15. The crystalline form of claim 14, wherein the crystalline form is further characterized by having XRPD diffraction peaks (2θ degrees) at 23.8°±0.2°, 28.6°±0.2°, and 32.0°±0.2°.

16. The crystalline form of claim 14, wherein the crystalline form is further characterized by having XRPD diffraction peaks (2θ degrees) at 12.9°±0.2°, 19.1°±0.2°, 19.5°±0.2°, 21.2°±0.2°, 22.3°±0.2°, 36.4°±0.2°, 37.6°±0.2°, and 39.5°±0.2°.

17. The crystalline form of claim 14, wherein the crystalline form is prepared by:
mixing D-glyceric acid with a calcium salt to form a first solution;
adjusting pH of the first solution to a pH range;
adding methyl alcohol into the first solution to form a second solution;
refluxing the second solution at a first temperature for a first time period;
stopping refluxing, and stirring and cooling the second solution naturally;
after cooling the second solution to a second temperature, adding a solvent into the second solution and stirring to form a third solution;
aging the third solution for a second time period; and
filtering the third solution to obtain calcium glycerate sample and drying the calcium glycerate sample to obtain the crystalline form of calcium glycerate.

18. The crystalline form of claim 17, wherein
the pH range includes a range from 2.5 to 5.8 or from 3.0 to 4.0,
a volume ratio of methyl alcohol to the first solution ranges from 0.3 to 1 or from 0.5 to 0.8,
the first temperature ranges from 60° C. to 80° C. or from 65° C. to 78° C., or
the first time period ranges from 20 min to 120 min or from 40 min to 80 min.

19-21. (canceled)

22. A method for preparing an amorphous form of calcium glycerate of formula (III) 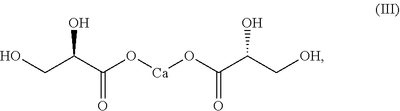
the method comprising:
stirring a crystalline form of calcium glycerate with a solvent at a first temperature for a first time period to form a suspension solution;
maintaining the first temperature and filtering the suspension solution to form a supersaturated solution;
stirring the supersaturated solution at a stirring rate, and cooling the supersaturated solution at a cooling rate;
after cooling the supersaturated solution to a second temperature, maintaining the second temperature, aging the supersaturated solution for a second time period, and filtering the supersaturated solution; and
drying the filtered product for a third time period at a pressure and a third temperature to obtain the amorphous form of calcium glycerate.

23. The method of claim 22, wherein
the first temperature ranges from 20° C. to 60° C. or from 40° C. to 50° C.,
the solvent includes at least one of methanol, ethanol, acetonitrile, acetone, dimethyl sulfoxide, or N—N dimethylformamide, or
the first time period ranges from 20 min to 120 min or from 60 min to 90 min.

24. The method of claim 22, wherein
the stirring rate ranges from 40 r/min to 120 r/min or from 60 r/min to 100 r/min, or
the cooling rate ranges from 40° C./h to 90° C./h or from 60° C./h to 80° C./h.

25. The method of claim 22, wherein
the second temperature ranges from −10° C. to 10° C./h or from 0° C. to 8° C.,
the second time period ranges from 1 h to 10 h or from 3 h to 5 h.

26. The method of claim 22, wherein
the pressure ranges from −0.07 MPa to 0.10 MPa or from −0.08 MPa to 0.09 MPa,
wherein the third temperature ranges from 20° C. to 50° C. or from 30° C. to 40° C., or
the third time period ranges from 1 h to 8 h or from 3 h to 5 h.

27. The method of claim 22, wherein the crystalline form of calcium D-glycerate has crystalline Formula:

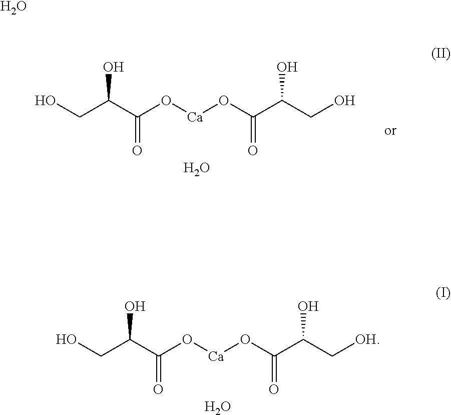

28-44. (canceled)
